SEQUENCE LISTING (5 sequences)

SEQ ID NO: 1 - DNA, 11 bp, Artificial
  Example of sequence for current application.
ctcttcnnnnn

SEQ ID NO: 2 - DNA, 11 bp, Artificial
  Example of sequence for current application.
nnnnngaagag

SEQ ID NO: 3 - DNA, 20 bp, artificial
  Example of sequence for current application.
ttaccctcactaaagggaga

SEQ ID NO: 4 - DNA, 40 bp, artificial
  Example of sequence for current application.
ggcacatcaattaccctcacuuuuuuuuuuuuuuuuuuuu

SEQ ID NO: 5 - DNA, 40 bp, artificial; identical to SEQ ID NO: 4
  Example of sequence for current application.

(19) United States
(12) Patent Application Publication
(10) Pub. No.: US 2004/0110153 A1
(43) Pub. Date: Jun. 10, 2004

(54) Compleixity management of genomic DNA by semi-specific amplification

(75) Inventors: Shoulian Dong, San Jose, CA (US); Xing Su, Cupertino, CA (US)

Correspondence Address: AFFYMETRIX, INC, ATTN: CHIEF IP COUNSEL, LEGAL DEPT., 3380 CENTRAL EXPRESSWAY, SANTA CLARA, CA, 95051, US

(73) Assignee: Affymetrix, INC., Santa Clara, CA

(21) Appl. No.: 10/316,811

(22) Filed: Dec. 10, 2002

(51) Int. Cl. C12Q 1/68; C12P 19/34

(52) U.S. Cl. 435/6; 435/91.2

(57) ABSTRACT

The presently claimed invention provides for novel methods and kits for reducing the complexity of a nucleic acid sample. In one embodiment specific fragments are amplified. The invention further provides for analysis of the above sample by hybridization to an array, which may be specifically designed to interrogate the desired fragments for particular characteristics, such as, for example, the presence or absence of a polymorphism.

### FIELD OF THE INVENTION

The invention relates to enrichment and amplification of sequences from a nucleic acid sample. In one embodiment, the invention relates to enrichment and amplification of nucleic acids for the purpose of further analysis. The present invention relates to the fields of molecular biology and genetics.

### BACKGROUND OF THE INVENTION

The past years have seen a dynamic change in the ability of science to comprehend vast amounts of data. Pioneering technologies such as nucleic acid arrays allow scientists to delve into the world of genetics in far greater detail than ever before. Exploration of genomic DNA has long been a dream of the scientific community. Held within the complex structures of genomic DNA lies the potential to identify, diagnose, or treat diseases like cancer, Alzheimer disease or alcoholism. Exploitation of genomic information from plants and animals may also provide answers to the world's food distribution problems.

Recent efforts in the scientific community, such as the publication of the draft sequence of the human genome in February 2001, have changed the dream of genome exploration into a reality. Genome-wide assays, however, must contend with the complexity of genomes; the human genome for example is estimated to have a complexity of 3×10⁹ base pairs. Novel methods of sample preparation and sample analysis that reduce complexity may provide for the fast and cost effective exploration of complex samples of nucleic acids, particularly genomic DNA.

### SUMMARY OF THE INVENTION

In one aspect of the invention, methods are provided for reducing the complexity of a first nucleic acid sample to produce a second nucleic acid sample wherein the second nucleic acid sample comprises a plurality of target sequences. The steps of the method comprise: fragmenting a first nucleic acid sample to create a population of fragments; modifying the ends of the fragments to generate a population of modified fragments; hybridizing a first primer comprising a 5′ first common sequence and a 3′ region that is complementary to the modified end of the modified fragments; extending the first primer to generate a plurality of extended first primers that are complementary to the modified nucleic acid fragments and comprise the first common sequence; hybridizing a plurality of target specific primers to the extended first primers wherein each target primer comprises a second common sequence and each species of target primer comprises a region that is upstream of a region of interest in a target sequence from the plurality of target sequences; extending the plurality of target primers to generate a plurality of extended target primers wherein each extended target primer comprises the second common sequence at the 5′ end and the complement of the first common sequence at the 3′ end; and amplifying the plurality of extended target primers to generate the second nucleic acid sample using a first amplification primer comprising at least part of the first common sequence and a second amplification primer comprising at least part of the second common sequence.

In some embodiments the population of nucleic acid fragments is modified by adding a homopolymeric tail using a terminal transferase and the first primer comprises a region that is complementary to the homopolymeric tail. The homopolymeric tail may be poly(dA) and the first primer may comprise a region of poly(dT). The first common sequence may comprise an RNA polymerase promoter sequence and a third nucleic acid sample may be generated from the second nucleic acid sample by in vitro transcription. The step of fragmenting a first nucleic acid sample may comprise digestion with at least one restriction enzyme.

In some embodiments the sequences present in the second nucleic acid sample are predetermined by, for example, a computer system.

In some embodiments the method of further comprises: labeling the second nucleic acid sample with a detectable label; hybridizing the second nucleic acid sample to an array of probes designed to interrogate one or more target sequences in the plurality of target sequences; generating a hybridization pattern; and analyzing the hybridization pattern to determine the presence or absence of the one or more target sequences.

In some embodiments the method further comprising: labeling the second nucleic acid sample with a detectable label; hybridizing the second nucleic acid sample to an array of probes designed to interrogate the genotype of one or more SNPs in the plurality of target sequences; generating a hybridization pattern; and analyzing the hybridization pattern to determine the genotype of the one or more SNPs in the plurality of target sequences.

In some embodiments the target sequences comprise SNPs and the array of probes is designed to interrogate at least 1000, 10,000 or 100,000 SNPs.

In some embodiments the sample is hybridized to an array of tag probes. The sample is mixed with a plurality of tagged primers wherein each species of tagged primer comprises a unique tag sequence and a sequence that is complementary to a region immediately adjacent to a polymorphic base in a target sequence and the plurality of tagged primers is extended. The tagged primers may be extended by a single nucleotide that is complementary to the polymorphic base in the target sequence and the single nucleotide may comprise a detectable label. The detectable label may be a different label for each type of nucleotide. The array of probes may be complementary to a plurality of tagged primers and each species of tagged primer may hybridized to a discrete location on the array. In some embodiments a hybridization pattern is detected and the identity of the polymorphic base is determined from the hybridization pattern.

In some embodiments the first and second common sequences are at least 50% homologous and may be, for example, 50-90% homologous.

In one embodiment the plurality of target sequences comprises a collection of polymorphic sequences. And the second nucleic acid sample is genotyped by: labeling the second nucleic acid sample with a detectable label to generate a labeled second nucleic acid sample; hybridizing the labeled second nucleic acid sample to an array of probes designed to genotype polymorphisms in the collection of polymorphic sequences; and analyzing the resulting hybridization pattern to determine the genotype of at least one polymorphism in the collection of polymorphic sequences.

In one embodiment a method of genotyping at least one polymorphic position in a collection of target sequences is disclosed. The method comprises the steps of first generating a first nucleic acid sample that is enriched for a collection of target sequences wherein each target sequence comprises a polymorphic position by: fragmenting a nucleic acid population to create a plurality of nucleic acid fragments; modifying the ends of the fragments to add common sequences to the ends of the fragments; amplifying a subset of the fragments; hybridizing the fragments to a first array comprising a collection of probes wherein each probe is complementary to a target sequence in the collection of target sequences; removing unhybridized fragments; and eluting and collecting the hybridized fragments to obtain the first nucleic acid sample. The first nucleic acid sample is then used to make a second nucleic acid sample by: amplifying the first nucleic acid sample using primers complementary to the common sequences; and labeling the amplified sample to obtain the second nucleic acid sample. The second nucleic acid sample is the hybridized to a second array comprising a collection of probes capable of interrogating the genotype of one or more of the polymorphic positions in the collection of target. The hybridization pattern may be analyzed to determine the genotype of one or more of the polymorphic positions.

The ends of the fragments may be modified by adding a first homopolymeric tail to the 3′ end of the fragments using terminal transferase; and then the step of amplifying a subset of fragments may be by hybridizing a first primer to the first homopolymeric tail wherein the first primer comprises a first common priming site; extending the first primer; adding a second homopolymeric tail to the 3′ end of the extended first primer; annealing a second primer to the second homopolymeric tail wherein the second primer comprises a second common priming site; extending the second primer to generate double stranded fragments; and amplifying the double stranded fragments using primers to the first and second common priming sites.

In some aspects of the invention the second homopolymeric tail is poly(A) and the second primer comprises a poly(U) region and the amplified fragments are treated with an enzyme that digests uridines. The enzyme may be uracil DNA glycosylase.

In another aspect of the invention a plurality of polymorphic regions is genotyped by hybridizing a plurality of polynucleotides to a nucleic acid population wherein each species of polynucleotide in the plurality of polynucleotides comprises in this order: a 5′ region that is complementary to a region that is immediately 5′ of a polymorphic region, a tag sequence, an optional priming site and a 3′ region that is complementary to a region immediately 3′ of a polymorphic region and including the polymorphic position, wherein each species of polynucleotide is complementary to a different polymorphic region; ligating the ends of the polynucleotides to create a population of circular polynucleotides; hybridizing a primer to the circular polynucleotides; extending the primer around the circular polynucleotides with a polymerase to generate copies of the circular polynucleotides; amplifying the copies of the circular polynucleotides; hybridizing the amplified copies to a genotyping array; and analyzing the hybridization pattern to determine the genotype of at least one of the polymorphic regions. A strand displacing polymerase, for example, Bst DNA polymerase, may be used for extending the primer. In some embodiments the primer is extended using a rolling circle amplification method.

In another aspect a nucleic acid population is enriched for target sequences, by hybridizing a plurality of primers to a nucleic acid population wherein each species of primer in the plurality of primers comprises in this order: a 3′ region that is complementary to a region immediately upstream of a polymorphic region, a tag sequence, a priming site and a 5′ region that is complementary to a region immediately downstream of the polymorphic region; extending at least one of the polynucleotides using the polymorphic regions as template; ligating the ends of the polynucleotides to create a population of circular polynucleotides; hybridizing a primer to the priming site in the circular polynucleotides; extending the primer around the circular polynucleotides with a polymerase to generate copies of the circular polynucleotides; and amplifying the copies of the circular polynucleotides using a primer to the tag sequence.

In another aspect a plurality of SNPs is genotyped by fragmenting a nucleic acid sample with a type IIs restriction enzyme; ligating an adaptor to the fragments; amplifying the adaptor ligated fragments using one target specific primer and a common primer that has a region that is complementary to the adaptor sequence and a selective region that is complementary to a subset of the possible sequences in the variable region of the type IIs cleavage site; fragmenting the amplified fragments; labeling the amplified fragments; hybridizing the amplified fragments to a genotyping array; and determining the genotype of at least one SNP in the plurality of SNPs.

In another aspect nucleic acid population is enriched for a plurality of target sequences by fragmenting a nucleic acid population to create a first population of fragments; hybridizing the population of modified fragments to an array of splint probes so that the 3′ and 5′ ends of the fragments are immediately adjacent; ligating the 3′ and 5′ ends of the fragments so that the fragments form a circular fragment; removing non-circular fragments; and amplifying the circular fragments.

In another aspect a nucleic acid population is enriched for a plurality of target sequences wherein each target sequence comprises a polymorphism by fragmenting a nucleic acid population to create a first population of nucleic acid fragments; adding a first common sequence to one end of the fragments and a second common sequence to the other end of the fragments to generate a population of modified fragments; hybridizing the modified fragments to an array comprising probes that are complementary to at least one target sequence in the plurality of target sequences; removing unhybridized fragments; bringing the 5′ and 3′ ends of the hybridized fragments together by hybridizing to the hybridized fragments a splint oligonucleotide that is complementary to at least part of the first sequence and at least part of the second sequence; ligating the ends of the hybridized fragments to create circular fragments; and amplifying the circular fragments.

### BRIEF DESCRIPTION OF THE FIGURES

FIG. 1 shows a schematic of genotyping by semi-specific amplification. Genomic fragments are modified so that they have a common priming sequence incorporated downstream of a polymorphism and are then amplified with a primer to the common sequence and a primer to a region upstream of the polymorphism so that the polymorphism is amplified. Polymorphisms in the amplified fragments may be labeled and detected by hybridization to an array.

FIG. 2 shows a schematic of genotyping by the use of two arrays, a capture array and an analysis array. A reduced complexity sample of genomic DNA is first hybridized to an array that is designed to hybridize to a selected group of target nucleic acids. Non-hybridized nucleic acids are removed by washing. The hybridized nucleic acids are eluted from the array, amplified, labeled and hybridized to an array designed to genotype polymorphisms.

FIG. 3A shows a method for incorporating common priming sites on the ends of a population of fragments and hybridizing to a capture array. One of the priming sites can be modified by the addition of one or more uridines and subsequently cleaved with uracil-DNA-glycosidase (UNG) to remove part of one of the strands so that the fragments are not entirely double stranded.

FIG. 3B shows a method for eluting fragments from a capture chip, amplifying and labeling the fragments and hybridizing the fragments to an array that interrogates SNPs. Double stranded fragments may be made partially single stranded by incorporation of uridines and digestion with uracil-DNA-glycosidase.

FIG. 4 shows a method for amplification of a collection of target nucleic acids using allele specific oligonucleotides that are complementary to a region immediately upstream and downstream of a polymorphism. The oligonucleotides are circularized and then amplified using rolling circle amplification.

FIG. 5 shows a method for amplification of a target nucleic acid using an allele specific oligonucleotide that is complementary to a region immediately upstream and downstream of a polymorphism. The oligonucleotide is circularized and then amplified with a first round of amplification using rolling circle amplification and a second round of amplification using primers to a common priming sequence.

FIG. 6 shows a method for enriching for a subset of target nucleic acids. Genomic DNA is digested with one or more Type IIs restriction enzymes and ligated to adaptors. Fragments are amplified with one primer that is specific for each target sequence to be amplified and a common primer to the adaptor.

FIG. 7 shows a schematic for enriching for a subset of fragments by hybridizing the fragments to an array of probes that are complementary to the ends of the fragments so that the fragments can be circularized. The splint probes are complementary to known sequences in the target fragments. Non-circularized fragments can then be removed and the circularized fragments are amplified.

FIG. 8 shows a schematic for enriching for a subset of fragments by hybridizing the fragments to an array of probes that are complementary to target sequences then bringing the ends of the target sequences together using a splint oligonucleotide so that the 5′ and 3′ ends of target sequences can be ligated. The fragments are modified by addition of common sequences at the 5′ and 3′ ends and the splint is complementary to the common sequences. Non-circularized fragments are removed and circularize fragments amplified.

### DETAILED DESCRIPTION OF THE PREFERRED EMBODIMENTS

(A) General

The present invention has many preferred embodiments and relies on many patents, applications and other references for details known to those of the art. Therefore, when a patent, application, or other reference is cited or repeated below, it should be understood that it is incorporated by reference in its entirety for all purposes as well as for the proposition that is recited.

As used in this application, the singular form “a,” “an,” and “the” include plural references unless the context clearly dictates otherwise. For example, the term “an agent” includes a plurality of agents, including mixtures thereof.

An individual is not limited to a human being but may also be other organisms including but not limited to mammals, plants, bacteria, or cells derived from any of the above.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. The same holds true for ranges in increments of 10⁵, 10⁴, 10³, 10², 10, 10⁻¹, 10⁻², 10⁻³, 10⁻⁴, or 10⁻⁵, for example. This applies regardless of the breadth of the range.

The practice of the present invention may employ, unless otherwise indicated, conventional techniques and descriptions of organic chemistry, polymer technology, molecular biology (including recombinant techniques), cell biology, biochemistry, and immunology, which are within the skill of the art. Such conventional techniques include polymer array synthesis, hybridization, ligation, and detection of hybridization using a label. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer (anyone have the cite), Gait, “Oligonucleotide Synthesis: A Practical Approach” 1984, IRL Press, London, Nelson and Cox (2000), Lehninger, Principles of Biochemistry 3^{rd} Ed., W. H. Freeman Pub., New York, N.Y. and Berg et al. (2002) Biochemistry, 5^{th} Ed., W. H. Freeman Pub., New York, N.Y. all of which are herein incorporated in their entirety by reference for all purposes.

The present invention can employ solid substrates, including arrays in some preferred embodiments. Methods and techniques applicable to polymer (including protein) array synthesis have been described in U.S. Ser. No. 09/536,841, WO 00/58516, U.S. Pat. Nos. 5,143,854, 5,242,974, 5,252,743, 5,324,633, 5,384,261, 5,424,186, 5,451,683, 5,482,867, 5,491,074, 5,527,681, 5,550,215, 5,571,639, 5,578,832, 5,593,839, 5,599,695, 5,624,711, 5,631,734, 5,795,716, 5,831,070, 5,837,832, 5,856,101, 5,858,659, 5,936,324, 5,968,740, 5,974,164, 5,981,185, 5,981,956, 6,025,601, 6,033,860, 6,040,193, 6,090,555, and 6,136,269, in PCT Applications Nos. PCT/US99/00730 (International Publication Number WO 99/36760) and PCT/US01/04285, and in U.S. patent applications Ser. Nos. 09/501,099 and 09/122,216 which are all incorporated herein by reference in their entirety for all purposes.

Patents that describe synthesis techniques in specific embodiments include U.S. Pat. Nos. 5,412,087, 6,147,205, 6,262,216, 6,310,189, 5,889,165 and 5,959,098 which are each incorporated herein by reference in their entirety for all purposes. Nucleic acid arrays are described in many of the above patents, but the same techniques are applied to polypeptide arrays.

The present invention also contemplates many uses for polymers attached to solid substrates. These uses include gene expression monitoring, profiling, library screening, genotyping, and diagnostics. Gene expression monitoring and profiling methods can be shown in U.S. Pat. Nos. 5,800,992, 6,013,449, 6,020,135, 6,033,860, 6,040,138, 6,177,248 and 6,309,822. Genotyping and uses therefore are shown in U.S. Ser. No. 10/013,598, and U.S. Pat. Nos. 5,856,092, 6,300,063, 5,858,659, 6,284,460, 6,361,947, 6,368,799 and 6,333,179 which are each incorporated herein by reference. Other uses are embodied in U.S. Pat. Nos. 5,871,928, 5,902,723, 6,045,996, 5,541,061, and 6,197,506 which are incorporated herein by reference.

The present invention also contemplates sample preparation methods in certain preferred embodiments. For example, see the patents in the gene expression, profiling, genotyping and other use patents above, as well as U.S. Ser. No. 09/854,317, U.S. Pat. Nos. 5,437,990, 5,215,899, 5,466,586, 4,357,421, and Gubler et al., 1985, Biochemica et Biophysica Acta, Displacement Synthesis of Globin Complementary DNA: Evidence for Sequence Amplification, each of which is incorporated herein by reference in its entirety.

Prior to or concurrent with analysis, the nucleic acid sample may be amplified by a variety of mechanisms, some of which may employ PCR. See, e.g., PCR Technology: Principles and Applications for DNA Amplification (Ed. H. A. Erlich, Freeman Press, New York, N.Y., 1992); PCR Protocols: A Guide to Methods and Applications (Eds. Innis, et al., Academic Press, San Diego, Calif., 1990); Mattila et al., Nucleic Acids Res. 19, 4967 (1991); Eckert et al., PCR Methods and Applications 1, 17 (1991); PCR (Eds. McPherson et al., IRL Press, Oxford); and U.S. Pat. Nos. 4,683,202, 4,683,195, 4,800,159 4,965,188, and 5,333,675, each of which is incorporated herein by reference in their entireties for all purposes. The sample may be amplified on the array. See, for example, U.S. Pat. No. 6,300,070 and U.S. patent application Ser. No. 09/513,300, which are incorporated herein by reference.

Other suitable amplification methods include the ligase chain reaction (LCR) (e.g., Wu and Wallace, Genomics 4, 560 (1989), Landegren et al., Science 241, 1077 (1988) and Barringer et al. Gene 89:117 (1990)), transcription amplification (Kwoh et al., Proc. Natl. Acad. Sci. USA 86, 1173 (1989) and WO88/10315), self-sustained sequence replication (Guatelli et al., Proc. Nat. Acad. Sci. USA, 87, 1874 (1990), WO/88/10315 and WO90/06995), selective amplification of target polynucleotide sequences (U.S. Pat. No. 6,410,276), consensus sequence primed polymerase chain reaction (CP-PCR) (U.S. Pat. No. 4,437,975), arbitrarily primed polymerase chain reaction (AP-PCR) (U.S. Pat. No. 5,413,909, 5,861,245) and nucleic acid based sequence amplification (NABSA). (See, U.S. Pat. Nos. 5,409,818, 5,554,517, and 6,063,603, each of which is incorporated herein by reference). Other amplification methods that may be used are described in, U.S. Pat. Nos. 5,242,794, 5,494,810, 4,988,617 and in U.S. Ser. No. 09/854,317, each of which is incorporated herein by reference.

Additional methods of sample preparation and techniques for reducing the complexity of a nucleic sample are described in Dong et al., Genome Research 11, 1418 (2001), in U.S. Pat. No. 6,361,947, 6,391,592 and U.S. patent application Ser. Nos. 09/512,300, 09/916,135, 09/920,491, 09/910,292, and 10/013,598, which are incorporated herein by reference in their entireties.

The present invention also contemplates detection of hybridization between ligands in certain preferred embodiments. See U.S. Pat. Nos. 5,143,854, 5,578,832; 5,631,734; 5,834,758; 5,936,324; 5,981,956; 6,025,601; 6,141,096; 6,185,030; 6,201,639; 6,218,803; and 6,225,625 and in PCT Application PCT/US99/06097 (published as WO99/47964), each of which also is hereby incorporated by reference in its entirety for all purposes.

The practice of the present invention may also employ conventional biology methods, software and systems. Computer software products of the invention typically include computer readable medium having computer-executable instructions for performing the logic steps of the method of the invention. Suitable computer readable medium include floppy disk, CD-ROM/DVD/DVD-ROM, hard-disk drive, flash memory, ROM/RAM, magnetic tapes and etc. The computer executable instructions may be written in a suitable computer language or combination of several languages. Basic computational biology methods are described in, e.g. Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2^{nd} ed., 2001).

The present invention may also make use of various computer program products and software for a variety of purposes, such as probe design, management of data, analysis, and instrument operation. See, U.S. Pat. Nos. 5,593,839, 5,795,716, 5,733,729, 5,974,164, 6,066,454, 6,090,555, 6,185,561, 6,188,783, 6,223,127, 6,229,911 and 6,308,170.

Additionally, the present invention may have preferred embodiments that include methods for providing genetic information over the internet. See U.S. patent applications and provisional application Nos. 10/063,559, 60/349,546, 60/376,003, 60/394,574, and 60/403,381

The present invention provides a flexible and scalable method for analyzing complex samples of nucleic acids, such as genomic DNA. These methods are not limited to any particular type of nucleic acid sample: plant, bacterial, animal (including human) total genome DNA, RNA, cDNA and the like may be analyzed using some or all of the methods disclosed in this invention. The word “DNA” may be used below as an example of a nucleic acid. It is understood that this term includes all nucleic acids, such as DNA and RNA, unless a use below requires a specific type of nucleic acid. This invention provides a powerful tool for analysis of complex nucleic acid samples. From experimental design to isolation of desired fragments and hybridization to an appropriate array, the invention provides for fast, efficient and inexpensive methods of complex nucleic acid analysis.

(B) Definitions

Nucleic acids according to the present invention may include any polymer or oligomer of pyrimidine and purine bases, preferably cytosine, thymine, and uracil, and adenine and guanine, respectively. (See Albert L. Lehninger, Principles of Biochemistry, at 793-800 (Worth Pub. 1982) which is herein incorporated in its entirety for all purposes). Indeed, the present invention contemplates any deoxyribonucleotide, ribonucleotide or peptide nucleic acid component, and any chemical variants thereof, such as methylated, hydroxymethylated or glucosylated forms of these bases, and the like. The polymers or oligomers may be heterogeneous or homogeneous in composition, and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states.

An oligonucleotide or polynucleotide is a nucleic acid ranging from at least 2, preferably at least 8, 15 or 20 nucleotides in length, but may be up to 50, 100, 1000, or 5000 nucleotides long or a compound that specifically hybridizes to a polynucleotide. Polynucleotides of the present invention include sequences of deoxyribonucleic acid (DNA) or ribonucleic acid (RNA) or mimetics thereof which may be isolated from natural sources, recombinantly produced or artificially synthesized. A further example of a polynucleotide of the present invention may be a peptide nucleic acid (PNA). (See U.S. Pat. No. 6,156,501 which is hereby incorporated by reference in its entirety.) The invention also encompasses situations in which there is a nontraditional base pairing such as Hoogsteen base pairing which has been identified in certain tRNA molecules and postulated to exist in a triple helix. “Polynucleotide” and “oligonucleotide” are used interchangeably in this application.

The term “fragment,” “segment,” or “DNA segment” refers to a portion of a larger DNA polynucleotide or DNA. A polynucleotide, for example, can be broken up, or fragmented into, a plurality of segments. Various methods of fragmenting nucleic acid are well known in the art. These methods may be, for example, either chemical or physical in nature. Chemical fragmentation may include partial degradation with a DNase; partial depurination with acid; the use of restriction enzymes; intron-encoded endonucleases; DNA-based cleavage methods, such as triplex and hybrid formation methods, that rely on the specific hybridization of a nucleic acid segment to localize a cleavage agent to a specific location in the nucleic acid molecule; or other enzymes or compounds which cleave DNA at known or unknown locations. Physical fragmentation methods may involve subjecting the DNA to a high shear rate. High shear rates may be produced, for example, by moving DNA through a chamber or channel with pits or spikes, or forcing the DNA sample through a restricted size flow passage, e.g., an aperture having a cross sectional dimension in the micron or submicron scale. Other physical methods include sonication and nebulization. Combinations of physical and chemical fragmentation methods may likewise be employed such as fragmentation by heat and ion-mediated hydrolysis. See for example, Sambrook et al., “Molecular Cloning: A Laboratory Manual,” 3rd Ed. Cold Spring Harbor Laboratory Press, Cold Spring Harbor, N.Y. (2001) (“Sambrook et al.) which is incorporated herein by reference for all purposes. These methods can be optimized to digest a nucleic acid into fragments of a selected size range. Useful size ranges may be from 100, 200, 400, 700 or 1000 to 500, 800, 1500, 2000, 4000 or 10,000 base pairs. However, larger size ranges such as 4000, 10,000 or 20,000 to 10,000, 20,000 or 500,000 base pairs may also be useful.

A number of methods disclosed herein require the use of restriction enzymes to fragment the nucleic acid sample. In general, a restriction enzyme recognizes a specific nucleotide sequence of four to eight nucleotides and cuts the DNA at a site within or a specific distance from the recognition sequence. For example, the restriction enzyme EcoRI recognizes the sequence GAATTC and will cut a DNA molecule between the G and the first A. The length of the recognition sequence is roughly proportional to the frequency of occurrence of the site in the genome. A simplistic theoretical estimate is that a six base pair recognition sequence will occur once in every 4096 (4⁶) base pairs while a four base pair recognition sequence will occur once every 256 (4⁴) base pairs. In silico digestions of sequences from the Human Genome Project show that the actual occurrences are even more infrequent, depending on the sequence of the restriction site. Because the restriction sites are rare, the appearance of shorter restriction fragments, for example those less than 1000 base pairs, is much less frequent than the appearance of longer fragments. Many different restriction enzymes are known and appropriate restriction enzymes can be selected for a desired result. (For a description of many restriction enzymes see, New England BioLabs Catalog which is herein incorporated by reference in its entirety for all purposes).

Type-IIs endonucleases are a class of endonuclease that, like other endonucleases, recognize specific sequences of nucleotide base pairs within a double stranded polynucleotide sequence. Upon recognizing that sequence, the endonuclease will cleave the polynucleotide sequence, generally leaving an overhang of one strand of the sequence, or “sticky end.” The Type-IIs endonucleases are unique because they generally do not require palindromic recognition sequences and they generally cleave outside of their recognition sites. For example, the Type-IIs endonuclease EarI recognizes and cleaves in the following manner:

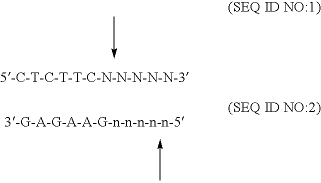

where the recognition sequence is -C-T-C-T-T-C-, N and n represent complementary, ambiguous base pairs and the arrows indicate the cleavage sites in each strand. As the example illustrates, the recognition sequence is non-palindromic, and the cleavage occurs outside of that recognition site.

Type-IIs endonucleases are generally commercially available and are well known in the art. Specific Type-IIs endonucleases which are useful in the present invention include, e.g., BbvI, BceAI, BfuAI, EarI, AlwI, BbsI, BsaI, BsmAI, BsmBI, BspMI, , HgaI, SapI, SfaNI, BsmFI, FokI, and PleI. Other Type-IIs endonucleases that may be useful in the present invention may be found, for example, in the New England Biolabs catalogue which is incorporated herein by reference in its entirety.

Adaptor sequences or adaptors are generally oligonucleotides of at least 5, 10, or 15 bases and preferably no more than 50 or 60 bases in length, however, they may be even longer, up to 100 or 200 bases. Adaptor sequences may be synthesized using any methods known to those of skill in the art. For the purposes of this invention they may, as options, comprise templates for PCR primers, restriction sites and promoters. The adaptor may be entirely or substantially double stranded. The adaptor may be phosphorylated or unphosphorylated on one or both strands. Adaptors are particularly useful in one embodiment of the current invention if they comprise a substantially double stranded region and short single stranded regions which are complementary to the single stranded region created by digestion with a restriction enzyme. For example, when DNA is digested with the restriction enzyme EcoRI the resulting double stranded fragments are flanked at either end by the single stranded overhang 5′-AATT-3′, an adaptor that carries a single stranded overhang 5′-AATT-3′ will hybridize to the fragment through complementarity between the overhanging regions. This “sticky end” hybridization of the adaptor to the fragment may facilitate ligation of the adaptor to the fragment but blunt ended ligation is also possible.

Adaptors can be used to introduce complementarity between the ends of a nucleic acid. For example, if a double stranded region of DNA is digested with a single enzyme so that each of the ends of the resulting fragments is generated by digestion with the same restriction enzyme, both ends will have the same overhanging sequence. For example if a nucleic acid sample is digested with EcoRI both strands of the DNA will have at their 5′ ends a single stranded region, or overhang, of 5′-AATT-3′. A single adaptor that has a complementary overhang of 5′-AATT-3′ can be ligated to both ends of the fragment. Each of the strands of the fragment will have one strand of the adaptor ligated to the 5′ end and the second strand of the adaptor ligated to the 3′ end. The two strands of the adaptor are complementary to one another so the resulting ends of the individual strands of the fragment will be complementary.

A single adaptor can also be ligated to both ends of a fragment resulting from digestion with two different enzymes. For example, if the method of digestion generates blunt ended fragments, the same adaptor sequence can be ligated to both ends. Alternatively some pairs of enzymes leave identical overhanging sequences. For example, BglII recognizes the sequence 5′-AGATCT-3′, cutting after the first A, and BamHI recognizes the sequence 5′-GGATCC-3′, cutting after the first G; both leave an overhang of 5′-GATC-3′. A single adaptor with an overhang of 5′-GATC-3′ may be ligated to both digestion products.

Digestion with two or more enzymes can be used to selectively ligate separate adapters to either end of a restriction fragment. For example, if a fragment is the result of digestion with EcoRI at one end and BamHI at the other end, the overhangs will be 5′-AATT-3′ and 5′GATC-3′, respectively. An adaptor with an overhang of AATT will be preferentially ligated to one end while an adaptor with an overhang of GATC will be preferentially ligated to the second end.

Methods of ligation will be known to those of skill in the art and are described, for example in Sambrook et at. and the New England BioLabs catalog both of which are incorporated herein by reference for all purposes. Methods include using T4 DNA Ligase which catalyzes the formation of a phosphodiester bond between juxtaposed 5′ phosphate and 3′ hydroxyl termini in duplex DNA or RNA with blunt or and sticky ends; Taq DNA ligase which catalyzes the formation of a phosphodiester bond between juxtaposed 5′ phosphate and 3′ hydroxyl termini of two adjacent oligonucleotides which are hybridized to a complementary target DNA; E.coli DNA ligase which catalyzes the formation of a phosphodiester bond between juxtaposed 5′-phosphate and 3′-hydroxyl termini in duplex DNA containing cohesive ends; and T4 RNA ligase which catalyzes ligation of a 5′ phosphoryl-terminated nucleic acid donor to a 3′ hydroxyl-terminated nucleic acid acceptor through the formation of a 3′−>5′ phosphodiester bond, substrates include single-stranded RNA and DNA as well as dinucleoside pyrophosphates; or any other methods described in the art.

A genome is all the genetic material of an organism. In some instances, the term genome may refer to the chromosomal DNA. Genome may be multichromosomal such that the DNA is cellularly distributed among a plurality of individual chromosomes. For example, in human there are 22 pairs of chromosomes plus a gender associated XX or XY pair. DNA derived from the genetic material in the chromosomes of a particular organism is genomic DNA. The term genome may also refer to genetic materials from organisms that do not have chromosomal structure. In addition, the term genome may refer to mitochondria DNA. A genomic library is a collection of DNA fragments representing the whole or a portion of a genome. Frequently, a genomic library is a collection of clones made from a set of randomly generated, sometimes overlapping DNA fragments representing the entire genome or a portion of the genome of an organism.

The term “chromosome” refers to the heredity-bearing gene carrier of a living cell which is derived from chromatin and which comprises DNA and protein components (especially histones). The conventional internationally recognized individual human genome chromosome numbering system is employed herein. The size of an individual chromosome can vary from one type to another with a given multi-chromosomal genome and from one genome to another. In the case of the human genome, the entire DNA mass of a given chromosome is usually greater than about 100,000,000 bp. For example, the size of the entire human genome is about 3×10⁹ bp. The largest chromosome, chromosome no. 1, contains about 2.4×10⁸ bp while the smallest chromosome, chromosome no. 22, contains about 5.3×10⁷ bp.

A chromosomal region is a portion of a chromosome. The actual physical size or extent of any individual chromosomal region can vary greatly. The term “region” is not necessarily definitive of a particular one or more genes because a region need not take into specific account the particular coding segments (exons) of an individual gene.

An allele refers to one specific form of a genetic sequence (such as a gene) within a cell, an individual or within a population, the specific form differing from other forms of the same gene in the sequence of at least one, and frequently more than one, variant sites within the sequence of the gene. The sequences at these variant sites that differ between different alleles are termed “variances”, “polymorphisms”, or “mutations”. At each autosomal specific chromosomal location or “locus” an individual possesses two alleles, one inherited from one parent and one from the other parent, for example one from the mother and one from the father. An individual is “heterozygous” at a locus if it has two different alleles at that locus. An individual is “homozygous” at a locus if it has two identical alleles at that locus.

The term genotyping refers to the determination of the genetic information an individual carries at one or more positions in the genome. For example, genotyping may comprise the determination of which allele or alleles an individual carries for a single SNP or the determination of which allele or alleles an individual carries for a plurality of SNPs. For example, a particular nucleotide in a genome may be an A in some individuals and a C in other individuals. Those individuals who have an A at the position have the A allele and those who have a C have the C allele. In a diploid organism the individual will have two copies of the sequence containing the polymorphic position so the individual may have an A allele and a C allele or alternatively two copies of the A allele or two copies of the C allele. Those individuals who have two copies of the C allele are homozygous for the C allele, those individuals who have two copies of the A allele are homozygous for the C allele, and those individuals who have one copy of each allele are heterozygous. The array may be designed to distinguish between each of these three possible outcomes. A polymorphic location may have two or more possible alleles and the array may be designed to distinguish between all possible combinations.

The term “target sequence”, “target nucleic acid” or “target” refers to a nucleic acid of interest. The target sequence may or may not be of biological significance. Typically, though not always, it is the significance of the target sequence which is being studied in a particular experiment. As non-limiting examples, target sequences may include regions of genomic DNA which are believed to contain one or more polymorphic sites, DNA encoding or believed to encode genes or portions of genes of known or unknown function, DNA encoding or believed to encode proteins or portions of proteins of known or unknown function, DNA encoding or believed to encode regulatory regions such as promoter sequences, splicing signals, polyadenylation signals, etc. The number of sequences to be interrogated can vary, but preferably are from 1, 10, 100, or 1000, to 10,000, 100,000 or 1,000,000 target sequences.

The term subset or representative subset refers to a fraction of a genome. The subset may be 0.1, 1, 3, 5, 10, 25, 50 or 75% of the genome. The partitioning of fragments into subsets may be done according to a variety of physical characteristics of individual fragments. For example, fragments may be divided into subsets according to size, according to the particular combination of restriction sites at the ends of the fragment, or based on the presence or absence of one or more particular sequences.

An “array” comprises a support, preferably solid, with nucleic acid probes attached to the support. Preferred arrays typically comprise a plurality of different nucleic acid probes that are coupled to a surface of a substrate in different, known locations. These arrays, also described as “microarrays” or colloquially “chips” have been generally described in the art, for example, U.S. Pat. Nos. 5,143,854, 5,445,934, 5,744,305, 5,677,195, 5,800,992, 6,040,193, 5,424,186 and Fodor et al., Science, 251:767-777 (1991). Each of which is incorporated by reference in its entirety for all purposes.

Arrays may generally be produced using a variety of techniques, such as mechanical synthesis methods or light directed synthesis methods that incorporate a combination of photolithographic methods and solid phase synthesis methods. Techniques for the synthesis of these arrays using mechanical synthesis methods are described in, e.g., U.S. Pat. Nos. 5,384,261, and 6,040,193, which are incorporated herein by reference in their entirety for all purposes. Although a planar array surface is preferred, the array may be fabricated on a surface of virtually any shape or even a multiplicity of surfaces. Arrays may be nucleic acids on beads, gels, polymeric surfaces, fibers such as fiber optics, glass or any other appropriate substrate. (See U.S. Pat. Nos. 5,770,358, 5,789,162, 5,708,153, 6,040,193 and 5,800,992, which are hereby incorporated by reference in their entirety for all purposes.)

Arrays may be packaged in such a manner as to allow for diagnostic use or can be an all-inclusive device; e.g., U.S. Pat. Nos. 5,856,174 and 5,922,591 incorporated in their entirety by reference for all purposes.

Preferred arrays are commercially available from Affymetrix under the brand name GeneChip® and are directed to a variety of purposes, including genotyping and gene expression monitoring for a variety of eukaryotic and prokaryotic species. (See Affymetrix Inc., Santa Clara and their website at affymetrix.com.)

A genotyping array comprises probes or sets of probes that are specific for each predicted allele of a polymorphism. A genotyping array may be designed to interrogate the genotype of one or more SNP. For each SNP the array will comprise a set of probes that are a perfect match for each known allele of the SNP or possibly for all possible alleles of a given SNP. The array will also comprise appropriate control probes such as one or more mismatch probes, probes that differ from the perfect match probe by one position. Antisense probes and antisense mismatch probes may also be included on the array as well as other control probes. See also, U.S. Ser. No. 60/417,190 which is incorporated herein by reference in its entirety.

Hybridization probes are oligonucleotides capable of binding in a base-specific manner to a complementary strand of nucleic acid. Such probes include peptide nucleic acids, as described in Nielsen et al., Science 254, 1497-1500 (1991), and other nucleic acid analogs and nucleic acid mimetics. See U.S. patent application Ser. No. 08/630,427-filed Apr. 3, 1996.

Hybridizations are usually performed under stringent conditions, for example, at a salt concentration of no more than 1 M and a temperature of at least 25° C. For example, conditions of 5×SSPE (750 mM NaCl, 50 mM NaPhosphate, 5 mM EDTA, pH 7.4) and a temperature of 25-30° C. are suitable for allele-specific probe hybridizations. For stringent conditions, see, for example, Sambrook, Fritsche and Maniatis. “Molecular Cloning A laboratory Manual” 2^{nd} Ed. Cold Spring Harbor Press (1989) which is hereby incorporated by reference in its entirety for all purposes above.

A splint probe has a first region that is complementary to the 3′ end of a selected target sequence and a second region that is complementary to the 5′ end of the same target sequence. The first region is immediately 3′ of the second region so that when the fragment hybridizes to the splint probe the 3′ end of the fragment and the 5′ end of the fragment are adjacent to one another. The ends may be immediately adjacent so that with the addition of ligase the ends can be joined. This may be used to facilitate circularization of a single stranded target molecule. Splint probes may be free in solution or they may be attached to a solid support. A splint probe may have additional sequence attached to the 5′ or 3′ end.

Polymorphism refers to the occurrence of two or more genetically determined alternative sequences or alleles in a population. A polymorphic marker or site is the locus at which divergence occurs. Preferred markers have at least two alleles, each occurring at frequency of preferably greater than 1%, and more preferably greater than 10% or 20% of a selected population. A polymorphism may comprise one or more base changes, an insertion, a repeat, or a deletion. A polymorphic locus may be as small as one base pair. Polymorphic markers include restriction fragment length polymorphisms, variable number of tandem repeats (VNTR's), hypervariable regions, minisatellites, dinucleotide repeats, trinucleotide repeats, tetranucleotide repeats, simple sequence repeats, and insertion elements such as Alu. The first identified allelic form is arbitrarily designated as the reference form and other allelic forms are designated as alternative or variant alleles. The allelic form occurring most frequently in a selected population is sometimes referred to as the wildtype form. Diploid organisms may be homozygous or heterozygous for allelic forms. A diallelic polymorphism has two forms. A triallelic polymorphism has three forms. A polymorphism between two nucleic acids can occur naturally, or be caused by exposure to or contact with chemicals, enzymes, or other agents, or exposure to agents that cause damage to nucleic acids, for example, ultraviolet radiation, mutagens or carcinogens.

Single nucleotide polymorphisms (SNPs) are positions at which two alternative bases occur at appreciable frequency (>1%) in the human population, and are the most common type of human genetic variation. The site is usually preceded by and followed by highly conserved sequences of the allele (e.g., sequences that vary in less than 1/100 or 1/1000 members of the populations).

A single nucleotide polymorphism usually arises due to substitution of one nucleotide for another at the polymorphic site. A transition is the replacement of one purine by another purine or one pyrimidine by another pyrimidine. A transversion is the replacement of a purine by a pyrimidine or vice versa. Single nucleotide polymorphisms can also arise from a deletion of a nucleotide or an insertion of a nucleotide relative to a reference allele.

An individual is not limited to a human being, but may also include other organisms including but not limited to mammals, plants, bacteria or cells derived from any of the above.

A tag or tag sequence is a selected nucleic acid with a specified nucleic acid sequence. A tag probe has a region that is complementary to a selected tag. A set of tags or a collection of tags is a collection of specified nucleic acids that may be of similar length and similar hybridization properties, for example similar Tₘ. The tags in a collection of tags bind to tag probes with minimal cross hybridization so that a single species of tag in the tag set accounts for the majority of tags which bind to a given tag probe species under hybridization conditions. For additional description of tags and tag probes and methods of selecting tags and tag probes see U.S. Ser. No. 08/626,285 and EP/0799897, each of which is incorporated herein by reference in their entirety.

In silico digestion is a computer aided simulation of enzymatic digests accomplished by searching a sequence for restriction sites. In silico digestion provides for the use of a computer system to model enzymatic reactions in order to determine experimental conditions before conducting any actual experiments. An example of an experiment would be to model digestion of the human genome with specific restriction enzymes to predict the sizes of the resulting restriction fragments.

(C.) Complexity Management

The present invention provides for novel methods of sample preparation and analysis involving managing or reducing the complexity of a nucleic acid sample, such as genomic DNA, in a predictable and reproducible manner by amplifying a representative subset of the sample. The invention further provides for analysis of the above subset by hybridization to an array. The array may be specifically designed with probes to the fragments predicted to be present in the amplified subset. In some embodiments the array may be specifically designed to interrogate the desired fragments for particular characteristics, such as, for example, the presence or absence of a polymorphism. In some embodiments the array is an array of probes to a collection of tag sequences. The invention is particularly useful when combined with other methods of genome analysis. As an example, the present techniques are useful to genotype individuals after polymorphisms have been identified. The invention discloses methods to amplify particular subsets of fragments and can be optimized to amplify fragments that contain identified polymorphisms.

SNPs are predicted to occur approximately once in every 1000 base pairs in the human genome. Large numbers of SNPs have been identified and are publicly available, for example on websites such as the SNP Consortium website (http://snp.cshl.org/). See, Altshuler et al., Science 407: 513-516 (2000) and The International SNP Map Working Group, Nature 409: 928-933 (2001) both of which are herein incorporated by reference in their entirety for all purposes. Amplification methods may be designed to amplify fragments that contain known SNPs and those amplified products may be analyzed to identify the genotype of a sample at one or more SNP locations. The methods provide for highly parallel analysis of a large number of SNPs, for example, more than 1,000, 5,000, 10,000 or 50,000, that are spaced throughout the genome. The SNPs may be selected for a number of desirable characteristics such as spacing throughout the genome, for example, location near known regions of interest in the genome, degree of polymorphism in a population or interest or in the general population, and empirical behavior of probe sets directed to individual SNPs. The methods also allow for highly parallel analysis of the same SNPs in large numbers of individuals. This provides a powerful tool for genetic mapping, linkage mapping and association analysis.

The present invention provides methods of complexity management of nucleic acid samples, such as genomic DNA. Many embodiments include the steps of: fragmenting the nucleic acid by digestion with one or more restriction enzymes or through alternative methods of fragmentation; amplifying a subset of the fragments using amplification conditions that preferentially amplify a predictable subset of fragments and hybridizing the amplified fragments to an array to detect the genotype of one or more polymorphisms. In a preferred embodiment the amplified sequences are exposed to an array which may have been specifically designed and manufactured to interrogate the amplified fragments. Design of both the complexity management steps and the arrays may be aided by computer modeling techniques. Generally, the steps of the present invention involve reducing the complexity of a nucleic acid sample using the disclosed techniques alone or in combination.

When interrogating genomes it is often useful to first reduce the complexity of the sample and analyze one or more subsets of the genome. Subsets can be defined by many characteristics of the fragments. In a preferred embodiment of the current invention, the subsets are defined by the presence of a polymorphic sequence. Collections of polymorphic sequences are targeted for amplification. In some embodiments a locus specific primer is used for each sequence to be amplified. Using a locus specific primer allows selection of the fragments and polymorphisms that will be amplified.

The genomic DNA sample of the current invention may be isolated according to methods known in the art, such as PCR, reverse transcription, and the like. It may be obtained from any biological or environmental source, including plant, animal (including human), bacteria, fungi or algae. Any suitable biological sample can be used for assay of genomic DNA. Convenient suitable samples include whole blood, tissue, semen, saliva, tears, urine, fecal material, sweat, buccal, skin and hair. In some embodiments the genomic DNA is fragmented. Any method of fragmentation may be used.

In many embodiments a collection of target sequences is analyzed. The collection may contain more than 1000, 5,000, 10,000, 50,000 or 100,000 different target sequences. In some embodiments a plurality of probes is used and each probe species is specific for a specific target sequence. In many embodiments target sequences contain or are predicted to contain a polymorphism, for example, a SNP. The polymorphism may be, for example, near a gene that is a candidate marker for a phenotype, useful for diagnosis of a disorder or for carrier screening or the polymorphism may define a haplotype block (see, Daly et al. Nat Genet. 29:229-32 (2001), and Rioux et al. Nat Genet. 29:223-8 (2001) and U.S. patent application Ser. No. 10/213,272, each of which is incorporated herein by reference in its entirety). A collection of probes may be designed so that each probe hybridizes near a polymorphism, for example, within 1, 5, 10, or 100 to 5, 10, 100, 1000, 10,000 or 100,000 bases of the polymorphism.

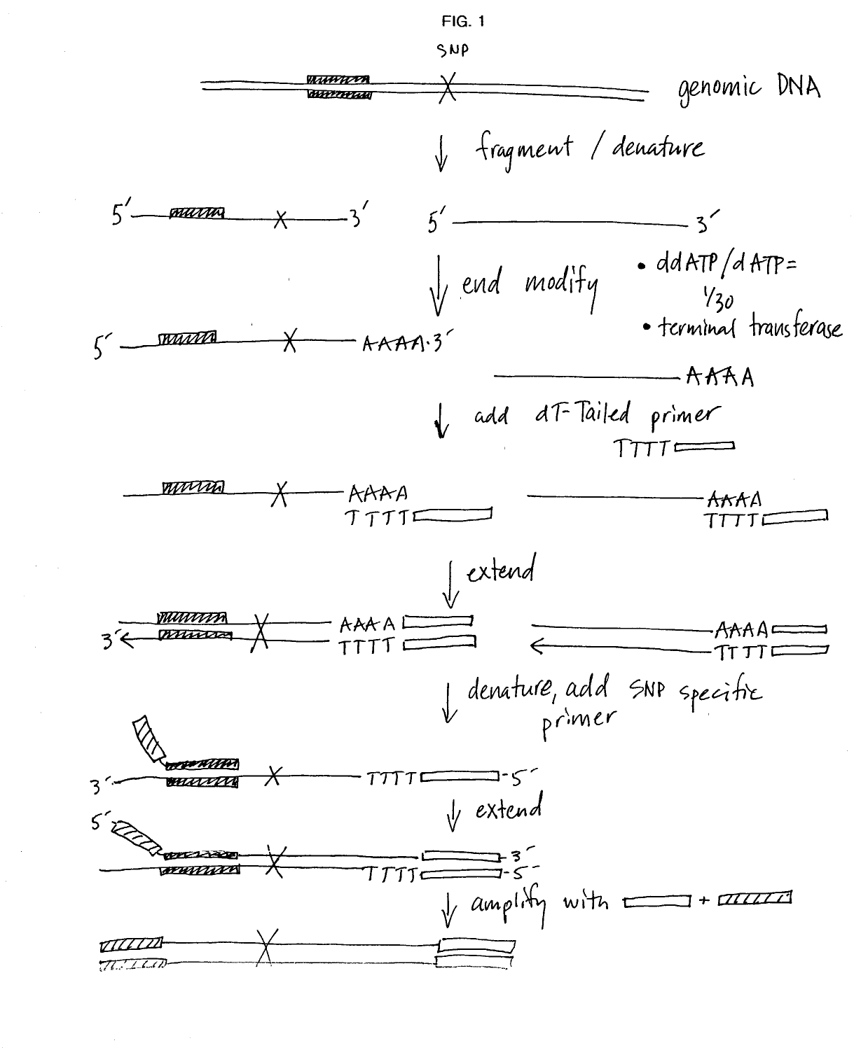

In a first embodiment (see FIG. 1) genomic DNA is fragmented and common primer sequences are added to the 3′ ends of the fragments by use of homopolymeric tailing. The homopolymeric tail serves as a primer binding site to initiate first round cDNA synthesis and locus specific primers that all share a common priming site are hybridized to the cDNA and extended. The fragments are then amplified using common primers. A genotyping array may be designed to interrogate the fragments.

The homopolymeric tail may be a poly(dA) tail added by, for example, terminal transferase. The length of the tail can be modulated by changing the ratio of dNTP to ddNTP. For example, in one embodiment the ration of ddATP:dATP is 1:30 so on average a ddATP will be incorporated once for every 30 dATPs incorporated. When a ddATP is incorporated in a fragment no additional dATPs will be added so the average length of the tails can be regulated. A poly(dT) primer may be hybridized to the newly added poly(A) tail of the fragments and cDNA may synthesized by extending the primer. In some embodiments the primer has a poly(dT) region at the 3′ end and a 5′ region that may be used as a common priming site. In some embodiments the primer may comprise a promoter for an RNA polymerase, such as, T7, T3 or SP6.

A selected subset of the cDNA is then made double stranded using a plurality of target specific primers wherein each species of primer has a locus specific region that is complementary to a region near a polymorphic sequence of interest. The primers are selected based on the target sequences that are to be amplified. This allows the complexity of the sample to be reduced in a reproducible and predictable manner, only those fragments that have been targeted with a locus specific primer will be efficiently amplified. The plurality of target specific primers also have a common priming site 5′ of the target specific region. The double stranded target fragments may then be amplified using a primer to the first common priming site on the poly(dT) primer and a primer to the second common priming site on the target specific primer. In some embodiments the first and second common priming sites are between 30 and 80% identical and primer-dimer amplification is reduced. In one embodiment they are about 50% identical.

The amplified target fragments may then be detected by hybridization to an array. In one embodiment the fragments are fragmented by, for example, DNase treatment, labeled with a detectable label such as a biotin labeled nucleotide, for example, biotin-ddATP, in the presence of terminal transferase and hybridized to an array designed to interrogate polymorphisms in the targeted sequences. In one embodiment the targeted sequences contain SNPs and the array has probes that are specific for each allele of the SNPs. The genotype of the SNP in the sample may be determined by analyzing the hybridization pattern on the array.

In some embodiments the amplified products are analyzed by hybridization to an array of probes attached to a solid support. In some embodiments an array of probes is specifically designed to interrogate a collection of target sequences. The array of probes may interrogate, for example, from 1,000, 5,000, 10,000 or 100,000 to 2,000, 5,000, 10,000, 100,000, 1,000,000 or 3,000,000 different target sequences. In one embodiment the target sequences contain SNPs and the array of probes is designed to interrogate the allele or alleles present at one or more polymorphic location. The array may comprise a collection of probes that hybridize specifically to one or more SNP containing sequences. The array may comprise probes that correspond to different alleles of the SNP. One probe or probe set may hybridize specifically to a first allele of a SNP, but not hybridize significantly to other alleles of the SNP and a second probe set may be designed to hybridize to a second allele of a SNP but not hybridize significantly to other alleles. A hybridization pattern from the array indicates which of the alleles are present in the sample. An array may contain probe sets to interrogate, for example, from 1,000, 5,000, 10,000 or 100,000 to 2,000, 5,000, 10,000, 100,000, 1,000,000 or 3,000,000 different SNPs.

In another embodiment an array of probes that are complementary to tag sequences is used to interrogate the target sequences. In some embodiments the amplified targets are analyzed on an array of tag sequences, for example, the Affymetrix GenFlex® array (Affymetrix, Inc., Santa Clara, Calif.). In this embodiment the primers comprise a tag sequence that is unique for each species of primer. A detectable label that is indicative of the allele present at the polymorphic site of interest is associated with the tag. The labeled tags are hybridized to the one or more arrays and the hybridization pattern is analyzed to determine which alleles are present.

In another embodiment the fragments are used as template in a single base extension reaction. The fragments are hybridized to a plurality of probes that end just 3′ of the polymorphic position. The probes are extended by a single nucleotide that is complementary to the polymorphic base. In some embodiments each species of probe also comprises a tag sequence. The extended probes are hybridized to an array of tag probes and the identity of the polymorphic base is detected.

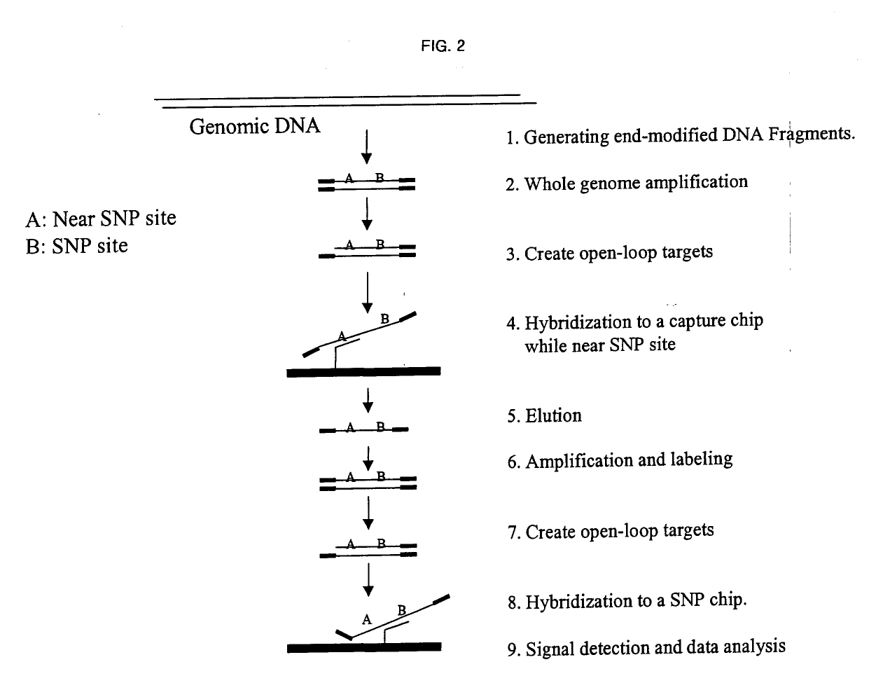

In another embodiment target sequences are first enriched by hybridization to a capture array and then genotyped by hybridization to a genotyping array (FIG. 2). The capture array is designed to hybridize to each of the target fragments but the probes may hybridize to any region of the target sequence and are not limited to short regions that contain a polymorphism. This allows optimization of probe design for uniform hybridization. Probes may also be spaced out throughout the entire length of the fragments. The genomic sample may first be fragmented, the fragments modified with one or more common sequences, for example, by ligation to common adaptor sequences, and the sample amplified with primers to the common adaptor sequences. This first amplification in some embodiments will result in a reduction in the complexity of the sample because not all of the fragments will be amplified with the same efficiency. The amplified sample may then by hybridized to an array that is designed to hybridize to a collection of target sequences. Non-target sequences that do not hybridize may be washed away resulting in a population that is enriched for target sequences. The hybridized sequences may then be amplified and hybridized to a second array that is designed to interrogate polymorphisms in the target sequences. The second array will comprise probes that are specific for each expected allele of a polymorphism in a target sequence as well as controls to determine specificity of hybridization.

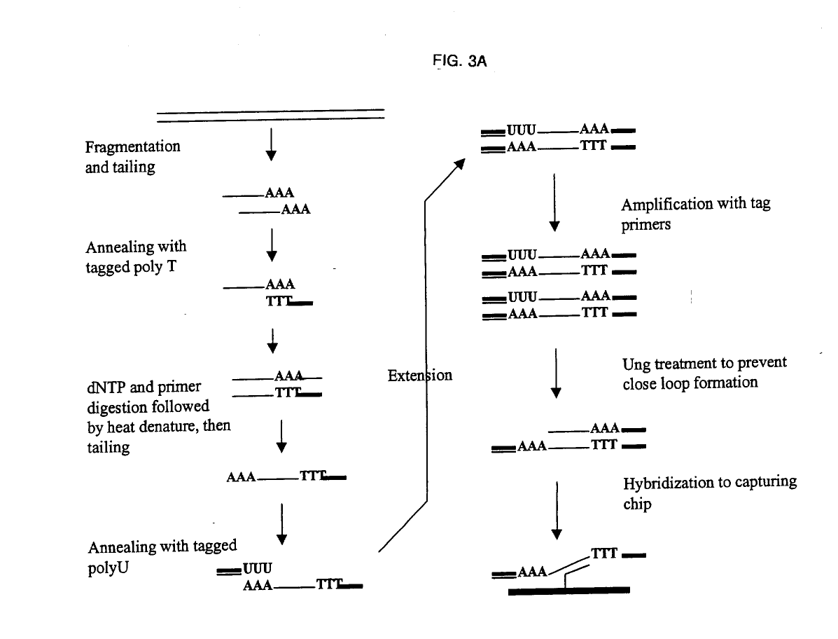

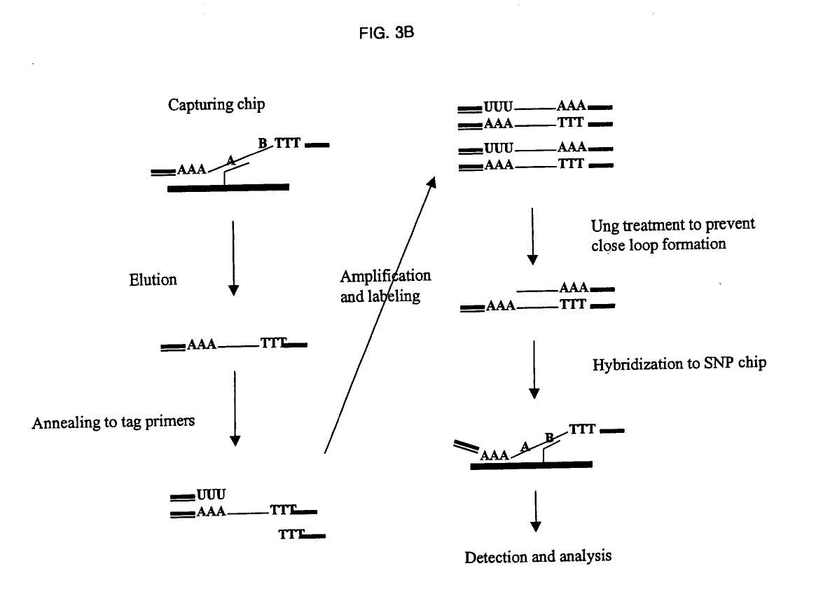

In some embodiments the common sequences are added by homopolymeric tailing. One embodiment is shown in FIG. 3A. Fragments are modified by the addition of a poly(A) tail, then a poly(T) primer with a common priming sequence is hybridized to the homopolymeric tail and extended to make cDNA. The cDNA is then modified by addition of a poly(A) tail. A poly(U) primer with a second priming site is hybridized to the poly(A) tail of the cDNA and extended resulting in a double stranded fragment flanked by common priming sites. The fragments have a poly(A):poly(U) duplex at one end and a poly(A):poly(T) duplex at the other end. The stretch of poly(U) can be used to facilitate cleavage by uracil-N-glycosidase prior to hybridization to a capture array. Cleavage removes one region of complementarity and may facilitate subsequent hybridization.

After hybridization to a capture array the fragments may be eluted and amplified using a primer that is complementary to one of the common priming sites and has a stretch of poly(U) and a primer that is complementary to the other common priming site and has a stretch of poly(T) (FIG. 3B). The fragments may be labeled during amplification or after amplification. The amplified fragments may be treated with an enzyme to remove part of one of the strands. Enzymatic methods include, for example, use of uracil DNA glycosylase (UDG) or (UNG). UNG catalyzes the hydrolysis of DNA that contains deoxyuridine at the site the uridine is incorporated. Incorporation of one or more uridines in the primer followed by treatment with UNG will result in cleavage of the primer. This results in formation of a partially double stranded fragment instead of a completely double stranded fragment. The partially double stranded fragment may result in more efficiently hybridization to the array. A thermolabile UNG may also be used. The fragments may then be hybridized to a genotyping array with probes designed to interrogate polymorphisms in selected target sequences.

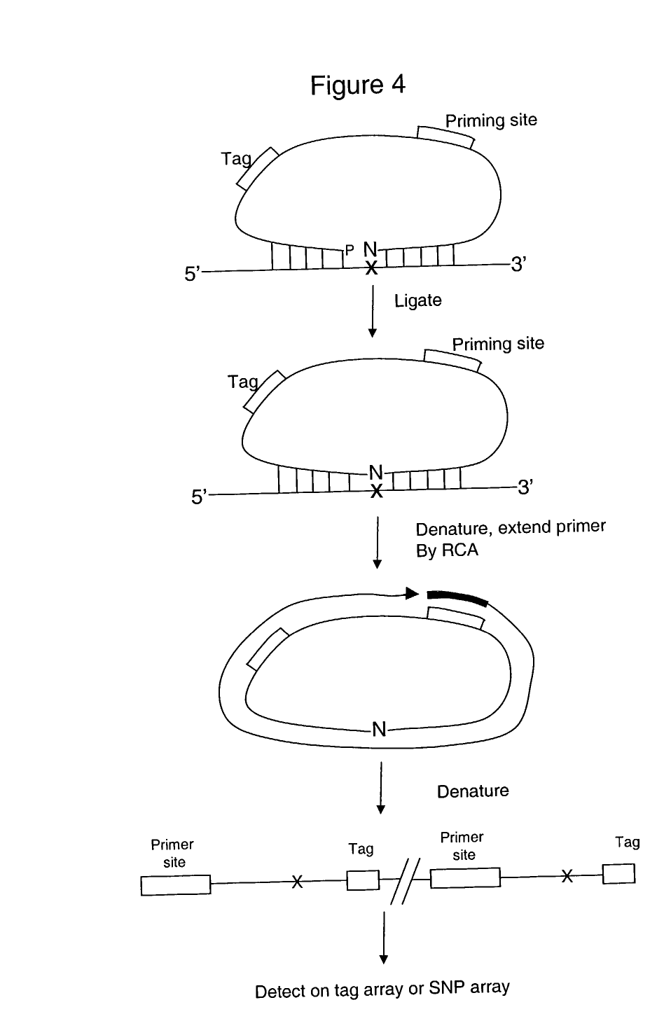

In another embodiment a circularizable probe is used for each target sequence. The probe has a 5′ region that is complementary to a region that is just 5′ of a polymorphic base and a 3′ region that is complementary to a region just 3′ of a polymorphic base, the 3′ terminal nucleotide of each probe is complementary to the polymorphic base and there is a different species of probe for each allele (FIG. 4). The probe hybridizes to the target sequence so that the 5′ and 3′ ends of the probe are juxtaposed. The juxtaposed ends may be ligated together to make a circular probe. Ligases that may be used include, for example, T4 DNA Ligase or Ampligase Thermostable DNA.

Uncircularized product may be removed, for example, by digestion with a nuclease such as Exonuclease VII or Exonuclease III. See, for example, U.S. Pat. No. 5,871,921 which is incorporated herein by reference. The circularized product will be resistant to nucleases that require either a free 5′ or 3′ end.

For each polymorphic position to be genotyped unique probes are designed for each expected allele. The final nucleotide in the probes (N) is the discrimination position and is complementary to one of the expected alleles. A stable hybrid between the probe and the target will form only when the 3′ terminal nucleotide of the probe is complementary to the polymorphic position in the target. Only probes that form a stable hybrid will be ligated. A generic primer that is complementary to a priming site may be used to amplify the probe sequence. The circularized probes may be amplified by rolling circle amplification using a common primer.

Rolling circle amplification is an isothermal amplification method utilizing a polymerase with strand displacement activity which generates many tandem copies of the complement to a circularized molecule, see, Lizardi et al., Nature Genet., 19, 225-232 (1997) which is incorporated herein by reference in its entirety. A single primer is hybridized to the single stranded circularized template and extended around the circle. The amplification primer hybridizes to the probe and is extended until it eventually displaces itself at its 5′ end once one complete revolution of the circularized probe is made. Continued polymerization and displacement results in a single stranded concatamer comprising multiple tandem repeats of the template. The fragments may be further amplified by, for example, PCR. In another embodiment a primer that is complementary to the tandem repeat copies is hybridized to the tandem repeat copies and extended to make the concatamers double stranded, see, Hafner et al. BioTechniques 30:852-867 (2001) which is incorporated herein by reference in its entirety.

The concatamer may be fragmented, and the fragments may be labeled and hybridized to an array of probes that detect individual alleles of a polymorphism. A set of probes may be designed to hybridize specifically to one allele of a SNP and a second set of probes may be designed to hybridize specifically to a second allele of the SNP. If the fragments hybridize to both sets of probes the individual is heterozygous at that position. If the fragments hybridize to only one set of probes the individual is homozygous for that SNP.

Polymerases with strand displacement activities included, for example, Klenow and Bst DNA polymerase, see, Hafner et al., BioTechniques 30:852-867 (2001) which is incorporated herein by reference in its entirety. T7 DNA Polymerase, Sequenase and φ29 polymerase may also be used.

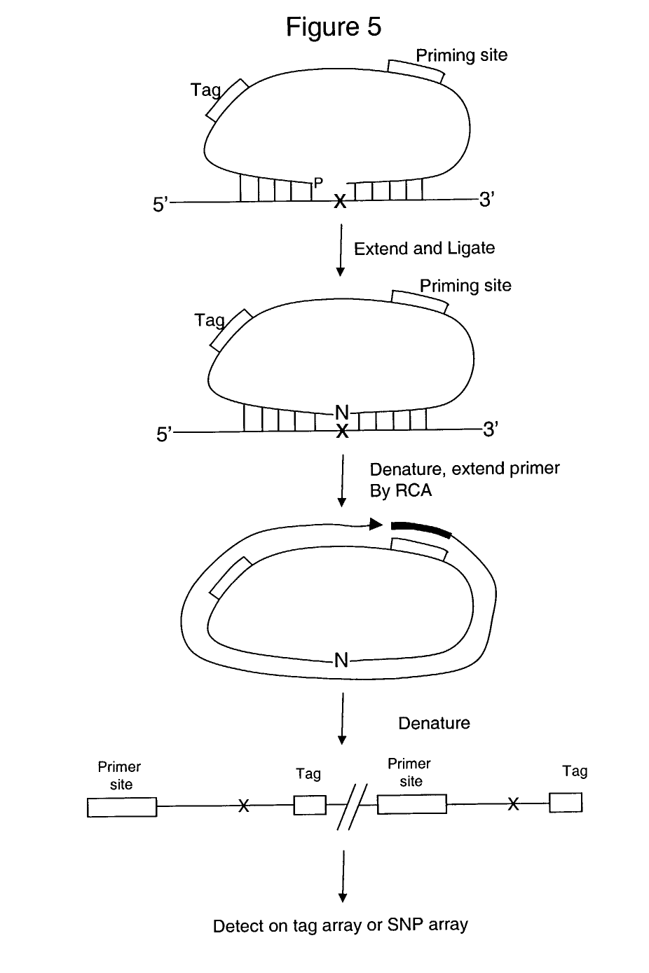

In another embodiment (FIG. 5) the ends are separated by a single base corresponding to the polymorphic base. The probe is extended at the 3′ end with a single base that is complementary to the polymorphic base, see, Lizardi et al., Nature Genet., 19, 225-232 (1997). In one embodiment four different extension reactions are used for each sample. Each of the reactions has a different nucleotide and each reaction is hybridized separately to an array. The hybridization pattern for each array is analyzed to determine what alleles are present.

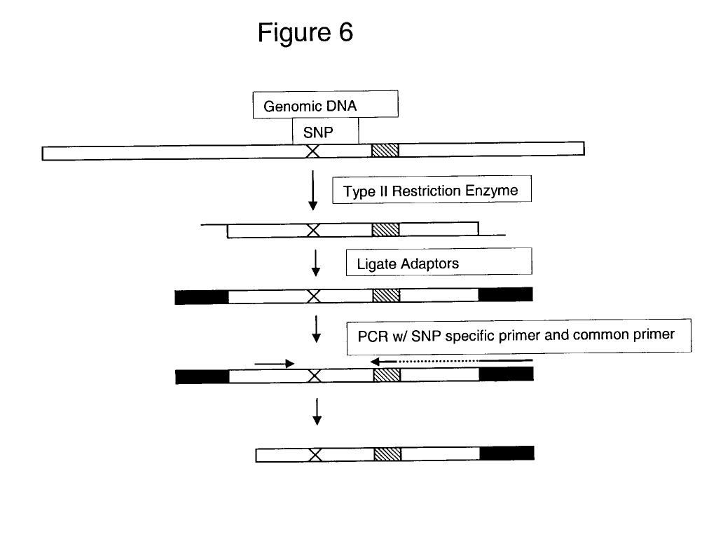

In another embodiment an adaptor with a type IIS restriction enzyme recognition site is ligated to genomic fragments (FIG. 6). The genomic DNA is first digested with one or more Type IIs enzymes and an adaptor is ligated to the fragments. Type IIs enzymes cleave downstream of their recognition sequence so the overhang that is left is variable. The region immediately upstream of the overhang may also be variable. A subset of the fragments is then amplified using a collection of target specific primers that hybridize upstream of the SNP and a common primer that hybridizes to at least part of the adaptor sequence, at least part of the variable region left by the type IIs enzyme and may also hybridize to the type IIs recognition sequence. The common primer may be designed so that only a selected subset of the fragments will be amplified by including only some of the possible sequences in the variable region. For example, if the type IIs enzyme leaves a variable region of four bases there are 256 possible sequences that might be present at that position. The primer can be designed to hybridize to only some of those sequences. One of the 4 bases could be constrained to a single base resulting in amplification of only approximately one quarter of the possible combinations.

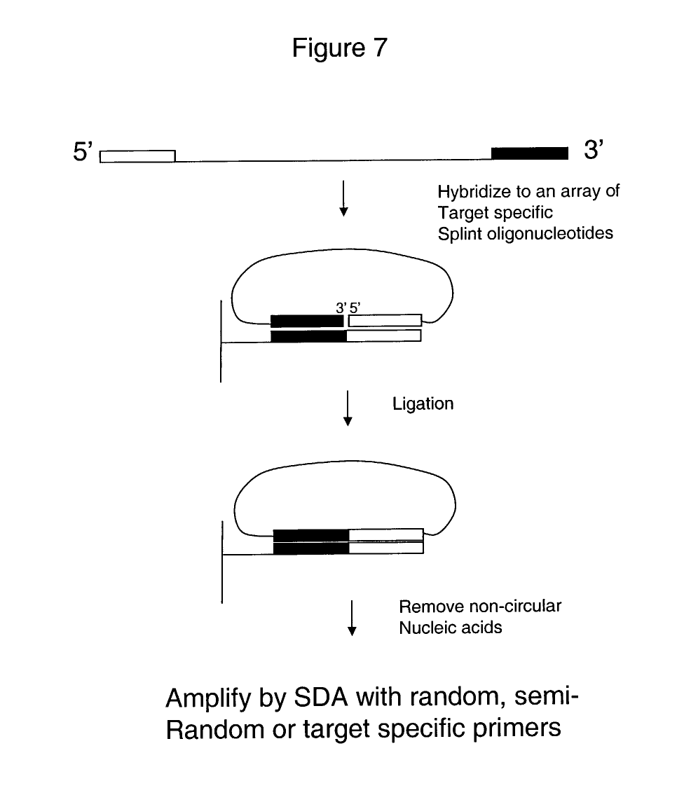

In another embodiment (FIG. 7) genomic DNA is fragmented and hybridized to an array of splint probes. The splint probes are complementary to known sequences at the 5′ and 3′ ends of target sequences. There is a unique species of splint probe for each target sequence to be amplified. Target sequences are hybridized to the splint probes on the array so that the 5′ and 3′ ends of the target sequence are juxtaposed and the ends are ligated together to form a circular target sequence. Non-circular nucleic acids may be removed and the circular target sequence may be amplified. Amplification may be primed by random primers, semi-random primers or target specific primers. In one embodiment amplification is by rolling circle amplification.

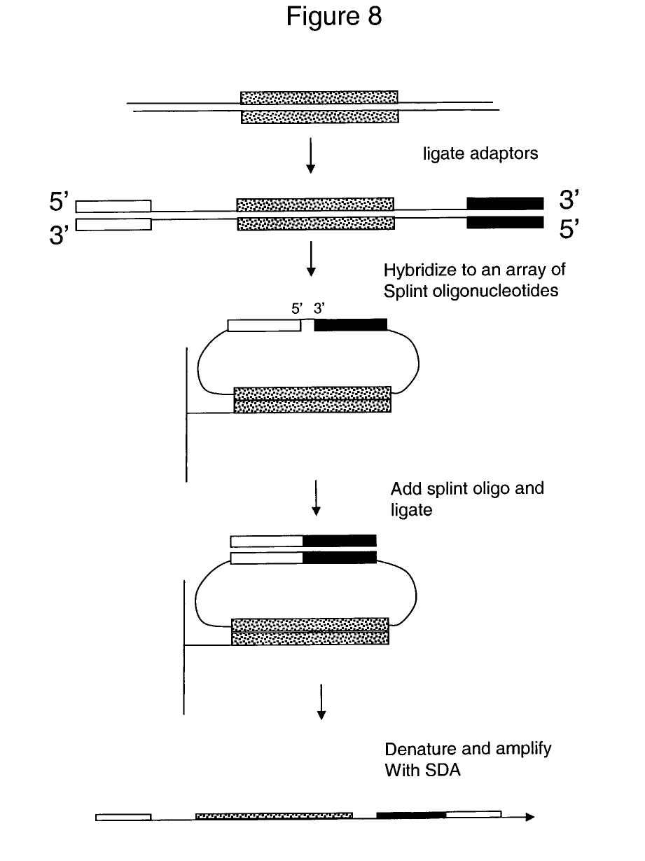

In another embodiment (FIG. 8) genomic DNA is fragmented and adaptors are ligated to the ends. The adaptors comprise a common sequence. The adaptor ligated fragments are hybridized to an array of target specific probes. Unhybridized fragments may be washed away. A splint oligonucleotide is then hybridized to the target sequences. The splint oligonucleotide is complementary to the adaptor sequences. Hybridization of the ends of the target sequences to the splint oligonucleotide results in juxtaposition of the 5′ and 3′ ends of the target sequences and the ends are then ligated together to form circular target sequences. The target sequences are then amplified using rolling circle amplification and a strand displacing polymerase.

There are many known methods of amplifying nucleic acid sequences including e.g., PCR. See, e.g., PCR Technology: Principles and Applications for DNA Amplification (ed. H. A. Erlich, Freeman Press, New York, N.Y., 1992); PCR Protocols: A Guide to Methods and Applications (eds. Innis, et al., Academic Press, San Diego, Calif., 1990); Mattila et al., Nucleic Acids Res. 19, 4967 (1991); Eckert et al., PCR Methods and Applications 1, 17 (1991); PCR (eds. McPherson et al., IRL Press, Oxford); and U.S. Pat. Nos. 4,683,202, 4,683,195, 4,800,159 4,965,188 and 5,333,675 each of which is incorporated herein by reference in their entireties for all purposes.

PCR is an extremely powerful technique for amplifying specific polynucleotide sequences, including genomic DNA, single-stranded cDNA, and mRNA among others. Various methods of conducting PCR amplification and primer design and construction for PCR amplification will be known to those of skill in the art. Generally, in PCR a double stranded DNA to be amplified is denatured by heating the sample. New DNA synthesis is then primed by hybridizing primers to the target sequence in the presence of DNA polymerase and excess dNTPs. In subsequent cycles, the primers hybridize to the newly synthesized DNA to produce discreet products with the primer sequences at either end. The products accumulate exponentially with each successive round of amplification.

The DNA polymerase used in PCR is often a thermostable polymerase. This allows the enzyme to continue functioning after repeated cycles of heating necessary to denature the double stranded DNA. Polymerases that are useful for PCR include, for example, Taq DNA polymerase, Tth DNA polymerase, Tfl DNA polymerase, Tma DNA polymerase, Tli DNA polymerase, and Pfu DNA polymerase. There are many commercially available modified forms of these enzymes including: AmpliTaq® and AmpliTaq Gold® both available from Applied Biosystems. Many are available with or without a 3- to 5′ proofreading exonuclease activity. See, for example, Vent® and Vent® (exo-) available from New England Biolabs.

Other suitable amplification methods include the ligase chain reaction (LCR) (e.g., Wu and Wallace, Genomics 4, 560 (1989) and Landegren et al., Science 241, 1077 (1988)), transcription amplification (Kwoh et al., Proc. Natl. Acad. Sci. USA 86, 1173 (1989)), and self-sustained sequence replication (Guatelli et al., Proc. Nat. Acad. Sci. USA, 87, 1874 (1990)) and nucleic acid based sequence amplification (NABSA). (See, U.S. Pat. Nos. 5,409,818, 5,554517, and 6,063,603). The latter two amplification methods include isothermal reactions based on isothermal transcription, which produce both single-stranded RNA (ssRNA) and double-stranded DNA (dsDNA) as the amplification products in a ratio of about 30 or 100 to 1, respectively.

As those of skill in the art will appreciate, after amplification, the resulting sequences may be further analyzed using any known method including sequencing, HPLC, hybridization analysis, cloning, labeling, etc.

A variety of nucleases may be used in one or more of the embodiments. Nucleases that are commercially available and may be useful in the present methods include: Mung Bean Nuclease, E. Coli Exonuclease I, Exonuclease III, Exonuclease VII, T7 Exonuclease, BAL-31 Exonuclease, Lambda Exonucl ease, RecJ_{f}, and Exonuclease T. Different nucleases have specificities for different types of nucleic acids making them useful for different applications. Exonuclease I catalyzes the removal of nucleotides from single-stranded DNA in the 3′ to 5′ direction. Exonuclease I degrades excess single-stranded primer oligonucleotide from a reaction mixture containing double-stranded extension products. Exonuclease III catalyzes the stepwise removal of mononucleotides from 3′-hydroxyl termini of duplex DNA. A limited number of nucleotides are removed during each binding event, resulting in coordinated progressive deletions within the population of DNA molecules. The preferred substrates are blunt or recessed 3′-termini, although the enzyme also acts at nicks in duplex DNA to produce single-strand gaps. The enzyme is not active on single-stranded DNA, and thus 3′-protruding termini are resistant to cleavage. The degree of resistance depends on the length of the extension, with extensions 4 bases or longer being essentially resistant to cleavage. This property can be exploited to produce unidirectional deletions from a linear molecule with one resistant (3′-overhang) and one susceptible (blunt or 5′-overhang) terminus. Exonuclease VII is a single-strand directed enzyme with 5′ to 3′- and 3′ to 5′-exonuclease activities making it the only bi-directional E. coli exonuclease with single-strand specificity. The enzyme has no apparent requirement for divalent cation, and is fully active in the presence of EDTA. Initial reaction products are acid-insoluble oligonucleotides which are further hydrolyzed into acid-soluble form. The products of limited digests are small oligomers (dimers to dodecamers). For additional information about nucleases see catalogues from manufacturers such as New England Biolabs, Beverly, Mass.

The materials for use in the present invention are ideally suited for the preparation of a kit suitable for obtaining a subset of a genome. Such a kit may comprise various reagents utilized in the methods, preferably in concentrated form. The reagents of this kit may comprise, but are not limited to, buffer, appropriate nucleotide triphosphates, appropriate dideoxynucleotide triphosphates, reverse transcriptases, nucleases, restriction enzymes, adaptors, ligases, DNA polymerases, primers and instructions for the use of the kit.

### Methods of Use

The methods of the presently claimed invention can be used for a wide variety of applications. Any analysis of genomic DNA may be benefited by a reproducible method of complexity management. Furthermore, the methods and enriched fragments of the presently claimed invention are particularly well suited for study and characterization of extremely large regions of genomic DNA.

In a preferred embodiment, the methods of the presently claimed invention are used for SNP discovery and to genotype individuals. For example, any of the procedures described above, alone or in combination, could be used to isolate the SNPs present in one or more specific regions of genomic DNA. Selection probes could be designed and manufactured to be used in combination with the methods of the invention to amplify only those fragments containing regions of interest, for example a region known to contain a SNP. Arrays could be designed and manufactured on a large scale basis to interrogate only those fragments containing the regions of interest. Thereafter, a sample from one or more individuals would be obtained and prepared using the same techniques which were used to prepare the selection probes or to design the array. Each sample can then be hybridized to an array and the hybridization pattern can be analyzed to determine the genotype of each individual or a population of individuals. Methods of use for polymorphisms and SNP discovery can be found in, for example, co-pending U.S. application Ser. Nos. 08/813,159 and 09/428,350 which are herein incorporated by reference in their entirety for all purposes).

Correlation of Polymorphisms with Phenotypic Traits

Most human sequence variation is attributable to or correlated with SNPs, with the rest attributable to insertions or deletions of one or more bases, repeat length polymorphisms and rearrangements. On average, SNPs occur every 1,000-2,000 bases when two human chromosomes are compared. (See, The International SNP Map Working Group, Science 409: 928-933 (2001) incorporated herein by reference in its entirety for all purposes.) Human diversity is limited not only by the number of SNPs occurring in the genome but further by the observation that specific combinations of alleles are found at closely linked sites.

Correlation of individual polymorphisms or groups of polymorphisms with phenotypic characteristics is a valuable tool in the effort to identify DNA variation that contributes to population variation in phenotypic traits. Phenotypic traits include physical characteristics, risk for disease, and response to the environment. Polymorphisms that correlate with disease are particularly interesting because they represent mechanisms to accurately diagnose disease and targets for drug treatment. Hundreds of human diseases have already been correlated with individual polymorphisms but there are many diseases that are known to have an, as yet unidentified, genetic component and many diseases for which a component is or may be genetic.

Many diseases may correlate with multiple genetic changes making identification of the polymorphisms associated with a given disease more difficult. One approach to overcome this difficulty is to systematically explore the limited set of common gene variants for association with disease.

To identify correlation between one or more alleles and one or more phenotypic traits, individuals are tested for the presence or absence of polymorphic markers or marker sets and for the phenotypic trait or traits of interest. The presence or absence of a set of polymorphisms is compared for individuals who exhibit a particular trait and individuals who exhibit lack of the particular trait to determine if the presence or absence of a particular allele is associated with the trait of interest. For example, it might be found that the presence of allele A1 at polymorphism A correlates with heart disease. As an example of a correlation between a phenotypic trait and more than one polymorphism, it might be found that allele A1 at polymorphism A and allele B1 at polymorphism B correlate with a phenotypic trait of interest.

Diagnosis of Disease and Predisposition to Disease

Markers or groups of markers that correlate with the symptoms or occurrence of disease can be used to diagnose disease or predisposition to disease without regard to phenotypic manifestation. To diagnose disease or predisposition to disease, individuals are tested for the presence or absence of polymorphic markers or marker sets that correlate with one or more diseases. If, for example, the presence of allele A1 at polymorphism A correlates with coronary artery disease then individuals with allele A1 at polymorphism A may be at an increased risk for the condition.

Individuals can be tested before symptoms of the disease develop. Infants, for example, can be tested for genetic diseases such as phenylketonuria at birth. Individuals of any age could be tested to determine risk profiles for the occurrence of future disease. Often early diagnosis can lead to more effective treatment and prevention of disease through dietary, behavior or pharmaceutical interventions. Individuals can also be tested to determine carrier status for genetic disorders. Potential parents can use this information to make family planning decisions.

Individuals who develop symptoms of disease that are consistent with more than one diagnosis can be tested to make a more accurate diagnosis. If, for example, symptom S is consistent with diseases X, Y or Z but allele A1 at polymorphism A correlates with disease X but not with diseases Y or Z an individual with symptom S is tested for the presence or absence of allele A1 at polymorphism A. Presence of allele A1 at polymorphism A is consistent with a diagnosis of disease X. Genetic expression information discovered through the use of arrays has been used to determine the specific type of cancer a particular patient has. (See, Golub et al. Science 286: 531-537 (2001) hereby incorporated by reference in its entirety for all purposes.)

Pharmacogenomics

Pharmacogenomics refers to the study of how genes affect response to drugs. There is great heterogeneity in the way individuals respond to medications, in terms of both host toxicity and treatment efficacy. There are many causes of this variability, including: severity of the disease being treated; drug interactions; and the individuals age and nutritional status. Despite the importance of these clinical variables, inherited differences in the form of genetic polymorphisms can have an even greater influence on the efficacy and toxicity of medications. Genetic polymorphisms in drug-metabolizing enzymes, transporters, receptors, and other drug targets have been linked to interindividual differences in the efficacy and toxicity of many medications. (See, Evans and Relling, Science 286: 487-491 (2001) which is herein incorporated by reference for all purposes).

An individual patient has an inherited ability to metabolize, eliminate and respond to specific drugs. Correlation of polymorphisms with pharmacogenomic traits identifies those polymorphisms that impact drug toxicity and treatment efficacy. This information can be used by doctors to determine what course of medicine is best for a particular patient and by pharmaceutical companies to develop new drugs that target a particular disease or particular individuals within the population, while decreasing the likelihood of adverse affects. Drugs can be targeted to groups of individuals who carry a specific allele or group of alleles. For example, individuals who carry allele A1 at polymorphism A may respond best to medication X while individuals who carry allele A2 respond best to medication Y. A trait may be the result of a single polymorphism but will often be determined by the interplay of several genes.

In addition some drugs that are highly effective for a large percentage of the population, prove dangerous or even lethal for a very small percentage of the population. These drugs typically are not available to anyone. Pharmacogenomics can be used to correlate a specific genotype with an adverse drug response. If pharmaceutical companies and physicians can accurately identify those patients who would suffer adverse responses to a particular drug, the drug can be made available on a limited basis to those who would benefit from the drug. See, for example, U.S. Pat. Nos. 6,033,860 and 6,333,155 which are incorporated herein by reference in their entirety.

Similarly, some medications may be highly effective for only a very small percentage of the population while proving only slightly effective or even ineffective to a large percentage of patients. Pharmacogenomics allows pharamaceutical companies to predict which patients would be the ideal candidate for a particular drug, thereby dramatically reducing failure rates and providing greater incentive to companies to continue to conduct research into those drugs.

Determination of Relatedness

There are many circumstances where relatedness between individuals is the subject of genotype analysis and the present invention can be applied to these procedures. Paternity testing is commonly used to establish a biological relationship between a child and the putative father of that child. Genetic material from the child can be analyzed for occurrence of polymorphisms and compared to a similar analysis of the putative father's genetic material. Determination of relatedness is not limited to the relationship between father and child but can also be done to determine the relatedness between mother and child, (see e.g. Staub et al., U.S. Pat. No.6,187,540) or more broadly, to determine how related one individual is to another, for example, between races or species or between individuals from geographically separated populations, (see for example H. Kaessmann, et al. Nature Genet. 22, 78 (1999)).

Forensics

The capacity to identify a distinguishing or unique set of forensic markers in an individual is useful for forensic analysis. For example, one can determine whether a blood sample from a suspect matches a blood or other tissue sample from a crime scene by determining whether the set of polymorphic forms occupying selected polymorphic sites is the same in the suspect and the sample. If the set of polymorphic markers does not match between a suspect and a sample, it can be concluded (barring experimental error) that the suspect was not the source of the sample. If the set of markers does match, one can conclude that the DNA from the suspect is consistent with that found at the crime scene. If frequencies of the polymorphic forms at the loci tested have been determined (e.g., by analysis of a suitable population of individuals), one can perform a statistical analysis to determine the probability that a match of suspect and crime scene sample would occur by chance. A similar comparison of markers can be used to identify an individual's remains. For example the U.S. armed forces collect and archive a tissue sample for each service member. If unidentified human remains are suspected to be those of an individual a sample from the remains can be analyzed for markers and compared to the markers present in the tissue sample initially collected from that individual.

Marker Assisted Breeding

Genetic markers can assist breeders in the understanding, selecting and managing of the genetic complexity of animals and plants. Agriculture industry, for example, has a great deal of incentive to try to produce crops with desirable traits (high yield, disease resistance, taste, smell, color, texture, etc.) as consumer demand increases and expectations change. However, many traits, even when the molecular mechanisms are known, are too difficult or costly to monitor during production. Readily detectable polymorphisms which are in close physical proximity to the desired genes can be used as a proxy to determine whether the desired trait is present or not in a particular organism. This provides for an efficient screening tool which can accelerate the selective breeding process.

### EXAMPLES

### Example 1

### Semi-Specific Amplification

Target sequences used were the human beta actin gene (X00351) and the Human GAPDH gene (M33179). One antisense primer was made for each target. The actin primer was complementary to sequence 1130-1111 and the GAPDH primer was complementary to sequence 1192-1173. Both primers are at least 80 nucleotides away from an intron site. A tag sequence was attached to each of the above primers the tag sequence was: ttaccctcactaaagggaga (SEQ ID NO:3). The common primers used were ST3U20: ggcacatcaattaccctcacuuuuuuuuuuuuuuuuuuuu (SEQ ID NO:4) and BT3: atcacacaattaccctcactaaagggaga (SEQ ID NO:5). The ST3U20 primer is used for copying tailed fragments and amplification and the BT3 primer is used for amplification and in vitro transcription.

The fragmentation reaction was 40 μl DNA (50 ng/μl), 9 μl water, 6 μl 10×fragmentation buffer, 3 μl MnCl₂ (25 mM) and 2 μl DNases (0.002 U/μl). Incubation was at 25° C. for 15 min, 95 ° C. for 10 min then to room temperature.

The end modification reaction or tailing reaction was 20 μl digested DNA, 0.7 μl water, 6 μl 5×TdT buffer (Promega), 1.8 μl CoCl2 (25 mM), 1 μl ddATP/dATP (33 uM/1000 μM) and 0.5 μl TdT (20 U/μl, Promega). Incubation was at 37° C. for 30 min, 95° C. for 5 min then to room temperature.

Tailed fragments were copied by mixing 5 μl tailed DNA, 22 μl water, 4 μl 10×PCR buffer II, 4 μl 25 mM MgCl2, 2 μl dNTP, 2 μl ST3U20 primer (10 μM) and 1 μl Klenow exo minus (5 U/μl). Incubation was for 30 min at 37° C., 5 min at 95° C. then to room temperature.

Targets were extended by mixing 40 ul copied fragments, 1 μl tagged actin primer (1 μl), 1 μl tagged GAPDH primer (1 μM), 1 μl thermo sequenase (3 U/μl). Incubation was at 95 for 20 sec, 55° C. for 2 min, and 72 ° C. for 1 min and this was repeated 9 times. Then 1 μl Exonuclease I (10 U/μl) was added and the mixture was incubated at 37° C. for 30 min, 95° C. for 5 min and then to room temperature.

For amplification 44 μl extended target was mixed with 2 μl BT3 (10 μM), 2 μl ST3U20(10 μM), 2 μl dNTP (2mM), 1 μl 10×PCR buffer II and 0.5 μl TaqGold (5 U/μl). Incubation was at 95° C. for 10 min, and 45 cycles of (95° C. for 20 sec, 60° C. for 20 sec and 72° C. for 20 sec) and then to room temperature.

For labeling 2.5 μl amplified DNA was mixed with 7.5 μl water, 7 μl NTP (11 mM, including biotin-CTP, Biotin-TTP), 1 μl T3 polymerase buffer (Ambion) and 2 μl T3 RNA polymerase (Ambion). This was incubated for 5 hours at 37° C. then 2 μl Proteinase K (25 μg/μl) was added and the mixture was incubated at 50° C. for 30 min.

The sample was hybridized to a Test 1 chip for 2 hours.

### Example 2

### Double Chip Method for SNP Genotyping

Genomic DNA is fragmented and denatured by heating to 95° C. The fragments are then tailed by incubation with ddATP/dATP and TdT. A biotinylated Tag1-T20 primer is hybridized to the fragments and extended to make cDNA. The reaction is treated with exoI digestion and shrimp phosphatase followed by heat inactivation of the enzymes. The cDNA is 3′ tailed with ddATP/dATP and TdT and extended with a Tag 2-U20 primer. Amplification is with Tag 2-U20 and Tag 1-T20. Amplified fragments were digested with UNG. The digested fragments are hybridized to capture chips. Hybridized fragments are eluted and amplified and the amplified fragments are hybridized to a genotyping array.

### Conclusion

From the foregoing it can be seen that the present invention provides a flexible and scalable method for analyzing complex samples of DNA, such as genomic DNA. These methods are not limited to any particular type of nucleic acid sample: plant, bacterial, animal (including human) total genome DNA, RNA, cDNA and the like may be analyzed using some or all of the methods disclosed in this invention. This invention provides a powerful tool for analysis of complex nucleic acid samples. From experiment design to isolation of desired fragments and hybridization to an appropriate array, the above invention provides for fast, efficient and inexpensive methods of complex nucleic acid analysis.

All publications and patent applications cited above are incorporated by reference in their entirety for all purposes to the same extent as if each individual publication or patent application were specifically and individually indicated to be so incorporated by reference. Although the present invention has been described in some detail by way of illustration and example for purposes of clarity and understanding, it will be apparent that certain changes and modifications may be practiced within the scope of the appended claims.

What is claimed is:

1. A method of reducing the complexity of a first nucleic acid sample to produce a second nucleic acid sample wherein said second nucleic acid sample comprises a plurality of target sequences, said method comprising:
fragmenting a first nucleic acid sample to create a population of fragments;
modifying the ends of the fragments to generate a population of modified fragments;
hybridizing to said modified fragments a first primer comprising a 5′ first common sequence and a 3′ region that is complementary to said modified fragments;
extending said first primer to generate a plurality of extended first primers that are complementary to said modified fragments and comprise said first common sequence;
hybridizing a plurality of target specific primers to the extended first primers wherein each target specific primer comprises a second common sequence and each species of target specific primer comprises a region that hybridizes to an extended first primer upstream of a region of interest in one of the target sequences;
extending said plurality of target specific primers to generate a plurality of extended target specific primers wherein each extended target specific primer comprises said second common sequence at the 5′ end and the complement of said first common sequence at the 3′ end; and
amplifying said plurality of extended target specific primers to generate said second nucleic acid sample using a first amplification primer comprising at least part of said first common sequence and a second amplification primer comprising at least part of said second common sequence.

2. The method of claim 1 wherein the fragments are modified by adding a homopolymeric tail using a terminal transferase and wherein said first primer comprises a region that is complementary to said homopolymeric tail.

3. The method of claim 2 wherein said homopolymeric tail is poly(dA) and said first primer comprises a region of poly(dT).

4. The method of claim 1 wherein said first common sequence comprises an RNA polymerase promoter sequence.

5. The method of claim 4 further comprising generating a third nucleic acid sample from said second nucleic acid sample by in vitro transcription.

6. The method of claim 1 wherein the step of fragmenting a first nucleic acid sample comprises digestion with at least one restriction enzyme.

7. The method of claim 1 wherein at least 50% of the sequences present in the second nucleic acid sample are predetermined.

8. The method of claim 7 wherein a computer system is used to predetermine sequences that will be present in the second nucleic acid sample.

9. The method of claim 1 wherein one or more sequences in said plurality of target sequences comprises a single nucleotide polymorphism.

10. The method of claim 1 further comprising:
labeling said second nucleic acid sample with a detectable label;
hybridizing said second nucleic acid sample to an array of probes designed to interrogate one or more target sequences in said plurality of target sequences;
generating a hybridization pattern; and
analyzing said hybridization pattern to determine the presence or absence of said one or more target sequences.

11. The method of claim 9 further comprising:
labeling said second nucleic acid sample with a detectable label;
hybridizing said second nucleic acid sample to an array of probes designed to interrogate the genotype of one or more SNPs in said plurality of target sequences;
generating a hybridization pattern; and
analyzing said hybridization pattern to determine the genotype of said one or more SNPs in said plurality of target sequences.

12. The method of claim 11 wherein said array of probes comprises probes capable of interrogating at least 1000 SNPs.

13. The method of claim 12 wherein said array of probes comprises probes capable of interrogating at least 10,000 SNPs.

14. The method of claim 13 wherein said array of probes comprises probes capable of interrogating at least 100,000 SNPs.

15. The method of claim 1 further comprising:
mixing said second nucleic acid sample with a plurality of tagged primers wherein each species of tagged primer comprises a unique tag sequence and a sequence that is complementary to a region immediately adjacent to a polymorphic base in a target sequence and extending said plurality of tagged primers.

16. The method of claim 15 wherein said tagged primers are extended by a single nucleotide that is complementary to the polymorphic base in said target sequence and wherein said single nucleotide comprises a detectable label.

17. The method of claim 16 further comprising:
hybridizing the extended tagged primers to an array of probes that are complementary to the tagged primers wherein each species of tagged primer hybridizes to an identifieable location on the array;
detecting a hybridization pattern; and
determining the identity of at least one polymorphic base.

18. The method of claim 1 wherein said first common sequence and said second common sequence are at least 50% homologous.

19. The method of claim 1 wherein said first common sequence and said second common sequence are about 50% to 90% homologous.

20. A method of genotyping a collection of polymorphic sequences comprising generating a second nucleic acid sample according to the method of claim 1 wherein said plurality of target sequences comprises a collection of polymorphic sequences;
labeling said second nucleic acid sample with a detectable label to generate a labeled second nucleic acid sample;
hybridizing said labeled second nucleic acid sample to an array of probes designed to genotype polymorphisms in said collection of polymorphic sequences; and
analyzing the resulting hybridization pattern to determine the genotype of at least one polymorphism in said collection of polymorphic sequences.

21. A method of genotyping at least one polymorphic position in a collection of target sequences, comprising:
(a) generating a first nucleic acid sample that is enriched for a collection of target sequences wherein each target sequence comprises a polymorphic position by:
fragmenting a nucleic acid population to create a plurality of nucleic acid fragments;
modifying the ends of the fragments to add common sequences to the ends of the fragments;
amplifying a subset of said fragments;
hybridizing said fragments to a first array comprising a collection of probes wherein each probe is complementary to a target sequence is said collection of target sequences;
removing unhybridized fragments; and
eluting and collecting the hybridized fragments to obtain said first nucleic acid sample;
(b) generating a second nucleic acid sample by:
amplifying said first nucleic acid sample using primers complementary to said common sequences; and
labeling the amplified sample to obtain said second nucleic acid sample;
(c) hybridizing said second nucleic acid sample to a second array comprising a collection of probes capable of interrogating the genotype of one or more of the polymorphic positions in said collection of target sequences; and
(d) analyzing the resulting hybridization pattern to determine the genotype of one or more of the polymorphic positions.

22. The method of claim 21 wherein said second array comprises probes capable of interrogating 1,000 or more genotypes.

23. The method of claim 22 wherein said second array comprises probes capable of interrogating 10,000 or more genotypes.

24. The method of claim 23 wherein said second array comprises probes capable of interrogating 100,000 or more genotypes.

25. The method of claim 21 wherein the step of modifying the ends of the fragments comprises: adding a first homopolymeric tail to the 3′ end of said fragments using terminal transferase; and the step of amplifying a subset of fragments comprises:
hybridizing a first primer to the first homopolymeric tail wherein said first primer comprises a first common priming site;
extending said first primer;
adding a second homopolymeric tail to the 3′ end of the extended first primer;
annealing a second primer to said second homopolymeric tail wherein said second primer comprises a second common priming site;
extending said second primer to generate double stranded fragments; and
amplifying said double stranded fragments using primers to said first and second common priming sites.

26. The method of claim 25 wherein said first common priming site comprises a tag sequence.

27. The method of claim 25 wherein said second common priming site comprises a tag sequence.

28. The method of claim 25 wherein said second homopolymeric tail is poly(A) and the second primer comprises a poly(U) region and further comprising the step of treating the amplified fragments with an enzyme that digests uridines.

29. The method of claim 28 wherein said enzyme is uracil DNA glycosylase.

30. A method for genotyping a plurality of polymorphic regions, comprising:
hybridizing a plurality of polynucleotides to a nucleic acid population wherein each species of polynucleotide in said plurality of polynucleotides comprises in this order: a 5′ region that is complementary to a region that is immediately 5′ of a polymorphic region, a tag sequence, an optional priming site and a 3′ region that is complementary to a region immediately 3′ of a polymorphic region and including the polymorphic position, wherein each species of polynucleotide is complementary to a different polymorphic region;
ligating the ends of said polynucleotides to create a population of circular polynucleotides;
hybridizing a primer to said circular polynucleotides;
extending said primer around said circular polynucleotides with a polymerase to generate copies of said circular polynucleotides;
amplifying said copies of said circular polynucleotides;
hybridizing said amplified copies to a genotyping array; and
analyzing the hybridization pattern to determine the genotype of at least one of the polymorphic regions.

31. The method of claim 30 wherein said polymerase is a strand displacing polymerase.

32. The method of claim 31 wherein said strand displacing polymerase is Bst DNA polymerase.

33. The method of claim 30, wherein the step of extending said primer is by rolling circle amplification.

34. The method of claim 30, wherein the step of amplifying said copies is done by PCR.

35. A method to enrich a nucleic acid population for target sequences, comprising:
hybridizing a plurality of primers to a nucleic acid population wherein each species of primer in said plurality of primers comprises in this order: a 3′ region that is complementary to a region immediately upstream of a polymorphic region, a tag sequence, a priming site and a 5′ region that is complementary to a region immediately downstream of said polymorphic region;
extending at least one of said polynucleotides using said polymorphic regions as template;
ligating the ends of said polynucleotides to create a population of circular polynucleotides;
hybridizing a primer to said priming site in said circular polynucleotides;
extending said primer around said circular polynucleotides with a polymerase to generate copies of said circular polynucleotides; and
amplifying said copies of said circular polynucleotides using a primer to said tag sequence.

36. The method of claim 35 wherein said polymerase is a strand displacing polymerase.

37. The method of claim 36 wherein said strand displacing polymerase is Bst DNA polymerase.

38. The method of claim 35, wherein the step of extending said primer is by rolling circle amplification.

39. A method for genotyping a plurality of SNPs comprising:
fragmenting a nucleic acid sample with a type IIs restriction enzyme;
ligating an adaptor to the fragments;
amplifying the adaptor ligated fragments using one target specific primer and a common primer that has a region that is complementary to the adaptor sequence and a selective region that is complementary to a subset of the possible sequences in the variable region of the type IIs cleavage site;
fragmenting the amplified fragments;
labeling the amplified fragments;
hybridizing the amplified fragments to a genotyping array; and
determining the genotype of at least one SNP in said plurality of SNPs.

40. A method to enrich a nucleic acid population for a plurality of target sequences comprising:
fragmenting a nucleic acid population to create a first population of fragments;
hybridizing the population of modified fragments to an array of splint probes so that the 3′ and 5′ ends of the fragments are immediately adjacent;
ligating said 3′ and 5′ ends of the fragments so that the fragments form a circular fragment;
removing non-circular fragments; and amplifying said circular fragments.

41. A method to enrich a nucleic acid population for a plurality of target sequences wherein each target sequence comprises a polymorphism, comprising:
fragmenting a nucleic acid population to create a first population of nucleic acid fragments;
adding a first common sequence to one end of the fragments and a second common sequence to the other end of the fragments to generate a population of modified fragments;
hybridizing the modified fragments to an array comprising probes that are complementary to at least one target sequence in said plurality of target sequences;
removing unhybridized fragments;
bringing the 5′ and 3′ ends of the hybridized fragments together by hybridizing to said hybridized fragments a splint oligonucleotide that is complementary to at least part of said first sequence and at least part of said second sequence;
ligating the ends of said hybridized fragments to create circular fragments; and
amplifying said circular fragments.

42. The method of claim 41, wherein amplifying is by rolling circle amplification.

43. The method of claim 41, wherein amplifying is done using a strand displacing polymerase.

44. The method of claim 43, wherein said strand displacing enzyme is Bst DNA polymerase.
